(19) United States
(12) Patent Application Publication
(10) Pub. No.: US 2019/0201307 A1
(43) Pub. Date: Jul. 4, 2019

(54) PERMANENT HAIRSTYLING COMPOSITION WITH BUNTE SALTS FROM AMINO ACIDS AND OLIGOPEPTIDES

(71) Applicant: Henkel AG & Co. KGaA, Duesseldorf (DE)

(72) Inventors: GEORG KNUEBEL, Duesseldorf (DE); THOMAS FOERSTER, Duesseldorf (DE); ROMAIN FLOUW, Tokyo (JP)

(73) Assignee: Henkel AG & Co. KGaA, Duesseldorf (DE)

(21) Appl. No.: 16/330,287

(22) PCT Filed: Jun. 30, 2017
(86) PCT No.: PCT/EP2017/066250

§ 371 Date: Mar. 4, 2019

(30) Foreign Application Priority Data
Sep. 5, 2016 (DE) 10 2016 216 761.8

(51) Int. Cl. A61K 8/44; A61Q 5/04; A61K 8/46; A61K 8/22; A45D 7/04

(52) U.S. Cl. CPC A61K 8/447; A61Q 5/04; A61K 8/46; A45D 2200/25; A45D 7/04; A61K 2800/87; A61K 2800/884; A61K 8/22

(57) ABSTRACT

A multicomponent packaging unit (kit of parts) for permanently changing the shape of keratinic fibers, in particular human hair, comprises the following packaged separately from one another:
a container (i) containing a cosmetic agent (A), and
a container (ii) containing a cosmetic agent (B),
   where
agent (A) in container (i) contains at least one keratin-reducing compound, and
agent (B) in container (ii) contains at least one compound of general formula (I)

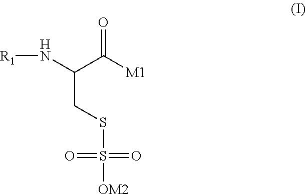

Also provides a method of using the kit and of using Bunte salts of formula (I) on hair.

### CROSS-REFERENCE TO RELATED APPLICATION

This application is a U.S. National-Stage entry under 35 U.S.C. § 371 based on International Application No. PCT/EP2017/066250, filed Jun. 30, 2017, which was published under PCT Article 21(2) and which claims priority to German Application No. 10 2016 216 761.8, filed Sep. 5, 2016, which are all hereby incorporated in their entirety by reference.

### TECHNICAL FIELD

The present disclosure is in the field of cosmetics and relates to a multicomponent packaging unit (kit of parts) for permanent change in the shape of keratinic fibers, said kit comprising at least two containers with at last two prepared separately from one another. The first preparation is a first cosmetic agent (A) containing at least one keratin-reducing compound. The second preparation is a second cosmetic agent (B) containing at least one Bunte salt of an amino acid or of an oligopeptide of formula (I).

### BACKGROUND

Another subject matter of the present disclosure is a method for permanent shaping of hair, in which the multicomponent packaging unit described above is used. Another subject matter of the present disclosure is the use of Bunte salts of formula (I) to reduce the hair damage caused by keratin-reducing compounds.

A permanently changing the shape of keratinic fibers is usually carried out in such a way that the fibers are shaped mechanically, the shape being set by suitable auxiliary means (for example, curlers or foil). Before and/or after shaping, the fibers are treated with an aqueous preparation of a keratin-reducing substance and rinsed with water or an aqueous solution after a treatment time. Following that, the fibers are then treated with the aqueous preparation of an oxidizing agent. After the treatment time, this agent is also rinsed out, and the fibers are freed of the mechanical shaping aids.

A permanent change in the shape of keratin fibers that will remain stable under the influence of water can be achieved only by a chemical treatment of hair aimed at cleaving the cystine-di sulfide bonds in the keratin of hair. This cleavage usually takes place under the influence of keratin-reducing substances. These are reducing agents that cleave some of the disulfide bonds of the keratin to form SH groups, thereby loosening the peptide crosslinking. There is a reorientation of the keratin structure due to the tension of the keratin fibers wound onto a curler. Disulfide bonds are formed anew under the influence of the oxidizing agent, and the keratin structure is newly set in the predetermined shape in this way. One such known method is a permanent wave treatment of human hair, which can be used to create curls and waves in straight hair.

Similarly, the agents described above may also be used for straightening curly and kinky hair. In this case, however, the mechanical shaping is accomplished by using a comb or a brush.

After the permanent treatment, the result is a loosened (or straightened) hair that is under tension internally, contains new amino acids (altered by reduction and/or oxidation) and is not completely crosslinked. This can result in a slow and gradual relaxation of shape, i.e., curls become looser. Another concomitant negative phenomenon of permanently waved hair is often damage to the hair structure because both the interior and the surface of hair become more hydrophilic due to the combination of the reduction step and the oxidation step. For this reason, high atmospheric humidity, frequent shampooing or combing and brushing of hair can cause the wave effect on damaged hair to be diminished. There is therefore still a search for novel agents and/or permanent wave methods with which all of these known disadvantages can be avoided.

In the past, there has been no lack of attempts to formulate permanent wave agents that will be gentle to the structure of the hair fiber, mild on the scalp and still supply an excellent permanent wave result. However, none of the agents known so far can be considered optimal.

### BRIEF SUMMARY

A multicomponent packaging unit (kit of parts) for permanently changing the shape of keratinic fibers, in particular human hair, comprises the following packaged separately from one another:
a container (i) comprising a cosmetic agent (A) and
a container (ii) comprising a cosmetic agent (B),

wherein
agent (A) in the container (i) comprises at least one keratin-reducing compound and
agent (B) in container (ii) comprises at least one compound of general formula (I)

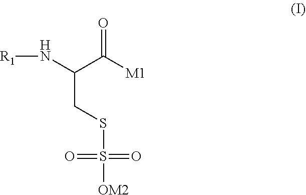

wherein

R1 stands for a hydrogen atom or for a structural element of formula (II)

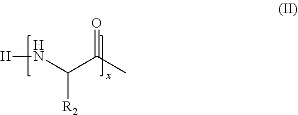

where

x stands for an integer from 1 to about 100,

the R2 radical in each one of the structural elements of formula (II) can each be selected independently of the preceding structural element of formula (II),

R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,

M1 stands for the —OM2 group or for a structural element of formula (III):

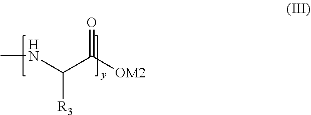

where

y stands for an integer from 1 to about 100,

the R3 radical in each of the structural elements of formula (III) can each be selected independently of the preceding structural element of formula (III),

R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,

M2 stands for a hydrogen atom, one equivalent of a monovalent or polyvalent cation, or an ammonium ion (NH₄)⁺.

A method for permanently changing the shape of keratinic fibers, comprises the following steps:

a1) Applying a cosmetic agent (A) to the keratinic fibers,

a2) Allowing the agent (A) to act for a period of from about 2 to about 60 minutes,

a3) Optionally rinsing the agent (A) out of the keratinic fibers,

b1) Applying a cosmetic agent (B) to the keratinic fibers,

b2) Allowing the agent (B) to act for a period of from about 2 to about 60 minutes,

b3) Optionally rinsing the agent (B) out of the keratinic fibers,

c1) Applying a cosmetic agent (C) to the keratinic fibers,

c2) Allowing the agent (C) to act for a period of from about 2 to about 60 minutes,

c3) Rinsing the agent (C) out of the keratinic fibers,

wherein
agent (A) is an agent as described above and further herein,
agent (B) is an agent as described above and further herein, and
agent (C) comprises an oxidizing agent.

In another embodiment, a method for reducing damage to hair caused by keratin-reducing compounds comprises applying to the hair a compound according to formula (I) as set forth herein.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure of discovering novel agents and/or novel methods for permanent shaping of keratinic fibers, these agents and/or methods to have an enhanced long-lasting permanent wave effect, while at the same time achieving the maximum possible protection of hair fibers.

It has now surprisingly been found that this object can be achieved optimally if the keratin fibers are permanently shaped by using products comprising not only the known keratin-reducing compounds, selected in particular from the group of thiols and/or disulfides, but also at least one Bunte salt of an amino acid, an oligopeptide and/or a peptide. The keratin-reducing compound and the Bunte salt here are made available and packaged separately in the form of at least two cosmetic agents that are offered to the user in the form of a multicomponent packaging unit.

The first subject matter of the present is a multicomponent packaging unit (kit of parts) for permanently changing the shape of keratinic fibers, in particular human hair, comprising the following packaged separately from one another:
a container (i) containing a cosmetic agent (A), and
a container (ii) containing a cosmetic agent (B),
   wherein
agent (A) in the container (i) contains at least one keratin-reducing compound, and
agent (B) in container (ii) contains at least one compound of the general formula (I):

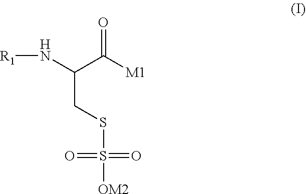

where
R1 stands for a hydrogen atom or for a structural element of the formula (II)

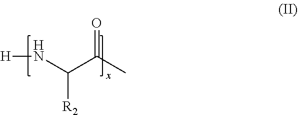

where
x stands for an integer from 1 to about 100,
the R2 radical in each of the structural elements of formula (II) can be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

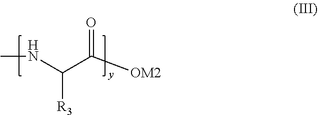

where
y stands for an integer from 1 to about 100,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group, and
M2 stands for a hydrogen atom, one equivalent of a monovalent or polyvalent cation, or an ammonium ion (NH₄)⁺.

Keratinic fibers, keratin-containing fibers or keratin fibers are understood to be furs, wool, feathers and in particular human hair. Although the agents as contemplated herein are suitable primarily for permanently changing the shape of keratin fibers, there is in principal nothing to prevent their use in other fields as well.

A permanent change in the shape of keratinic fibers is understood to refer to a change in shape in which first a reducing agent and then an oxidizing agent are applied to the keratin fibers. Due to the influence of the reducing agent—which is also referred to as a keratin-reducing compound—the reductive cleavage of the cystine-disulfide bonds in the hair takes place first. During the shaping process, the hair is converted to a new shape with the help of mechanical shaping aids (e.g., hair curlers, foil, etc.). The mechanical shaping of the hair may take place before, during or after the application of the reducing agent.

By subsequently applying the oxidizing agent, new disulfide bonds are formed in the shaped keratin fibers and the keratin fibers are secured in this way. The permanently changing the shape will usually survive several hair shampooings.

An agent for permanently changing the shape in the sense of the present disclosure may be used to both create curls (in this case, for example, hair curlers are used as the mechanical shaping aids) or may also be used for straightening (the mechanical shaping here takes place by employing a comb, for example).

The multicomponent packaging unit as contemplated herein comprises at least two separately packaged containers (i) and (ii). These containers may be tubes, bottles, sachets, bags or cans, for example, or any other container suitable for storing a cosmetic agent. The containers may be made of plastic, metal, glass or composites of these materials. The first container (i) contains a first cosmetic agent (A). The second container (ii) contains a second cosmetic agent (B).

In addition, the multicomponent packaging unit as contemplated herein may also comprise other containers, for example, a third container (iii) or a third and a fourth container (iii) and (iv). The third container (iii), which is optionally included, contains a third agent (C). The fourth container (iv), which is optionally included, contains a fourth cosmetic agent (D).

The agent (A) and the agent (B) (as well as optionally the agents (C) and (D)) contain the compounds that are essential for the present disclosure, each in a cosmetic vehicle, preferably in a suitable aqueous, alcoholic or aqueous-alcoholic vehicle. For the purposes of the hair treatment, such vehicles include creams, emulsions or gels, for example, or also foaming solutions that contain surfactants, such as shampoos, foam aerosols, foam formulations other preparations that are suitable for application to hair.

Aqueous alcoholic solutions in the sense of the present disclosure are understood to include aqueous solutions containing from about 3% to about 70% by weight of a C₁-C₄ alcohol, in particular ethanol and/or isopropanol. The agents as contemplated herein may additionally contain other organic solvents such as methoxybutanol, benzyl alcohol, ethyl diglycol or 1,2-propylene glycol. All water-soluble organic solvents are preferred. As contemplated herein, the aqueous vehicle contains at least about 30% by weight, in particular at least about 50% by weight water, based on the total weight of the agents. Aqueous vehicles are preferred.

### Keratin-Reducing Compounds in the Agent (A)

The agent (A) contains at least one keratin-reducing compound. Keratin-reducing compounds are understood to be the reducing agents whose reduction potential is high enough to achieve reductive cleavage of the cystine-disulfide bonds contained in the keratin fibers and to convert them to their corresponding thiols. The keratin-reducing compounds in the sense of the present disclosure are cosmetically tolerable.

The keratin-reducing compounds include in particular organic compounds having at least one thiol group (—SH) and/or at least one disulfide group (—S—S—).

The keratin-reducing compounds are thus organic compounds, which are characterized in that they contain as structural components a thiol or mercapto group (—SH), a disulfide group (—S—S—), or both.

In addition to the thiol group and/or the disulfide group, the keratin-reducing compounds may also have additional substituents, which may include one or more amino groups, hydroxy groups, acid groups, amide groups, or ester groups. The keratin-reducing compounds contained in the agent (A) do not fall under the group of Bunte salts (a) of formula (I) as contemplated herein but instead are different from this.

In other words, a first embodiment of the present disclosure is a multicomponent packaging unit (kit of parts) for permanently changing the shape of keratinic fibers, in particular human hair, comprising, packaged separately from one another:
a container (i) containing a cosmetic agent (A) and
a container (ii) containing a cosmetic agent (B),
   wherein
the agent (A) in container (i) contains at least one organic keratin-reducing compound with at least one thiol group (—SH) and/or at least one disulfide group (—S—S— group), and
the agent (B) in container (ii) contains at least one compound of general formula (I):

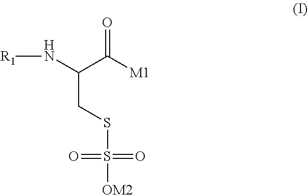

where
R1 stands for a hydrogen atom or for a structural element of formula (II)

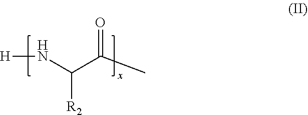

where
x stands for an integer from 1 to about 100,
the R2 radical in each of the structural elements of formula (II) may be selected independently of the previous structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

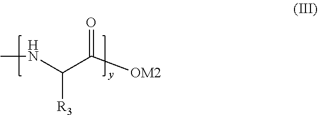

where
y stands for an integer from 1 to about 100, the radical R3 in each of the structural elements of formula (III) can be selected independently of the previous structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

Suitable keratin-reducing compounds include, for example, thioglycolic acid, thiolactic acid, thiomalic acid, cysteine, cysteamine, cystine, 2-mercaptoethane sulfonic acid and/or the physiologically tolerable salts of the aforementioned compounds. A physiologically tolerable salt is understood to be a salt of the aforementioned compounds, converted to its ionic form and having a physiologically tolerable counterion. A physiologically tolerable salt may be applied to hair and/or skin without any toxicological disadvantages, i.e., a physiologically tolerable salt of one of the aforementioned reducing agents does not have any more negative physiological effect than the reducing agent itself. Physiologically tolerable salts include, for example, the alkali metal salts, the alkaline earth metal salts or the ammonium salts of the aforementioned acids. A physiologically tolerable salt of cysteamine is, for example, its hydrochloride or hydrobromide.

In another especially preferred embodiment, a multicomponent packaging unit as contemplated herein is characterized in that the agent (A) in container (i) contains at least one keratin-reducing compound from the group of thioglycolic acid, thiolactic acid, thiomalic acid, cysteine, cysteamine, cystine, 2-mercaptoethane sulfonic acid and/or their physiologically tolerable salts.

Alternatively thioglycolic acid is also referred to as sulfanylacetic acid or mercaptoacetic acid and has CAS number 68-11-1. Suitable physiologically tolerable salts include, for example, the sodium salt, the potassium salt, or the ammonium salt of thioglycolic acid.

Ammonium thioglycolate is the ammonium salt of thioglycolic acid (i.e., the ammonium salt of sulfanylacetic acid (formula i)

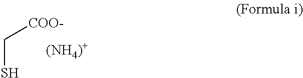

Thiolactic acid is understood to be D-thiolactic acid, L-thiolactic acid and/or a mixture thereof. Alternatively, thiolactic acid is also referred to as 2-mercaptopropionic acid and has CAS number 79-42-5. Suitable physiologically tolerable salts include, for example, the sodium salt, the potassium salt or the ammonium salt of thiolactic acid.

Ammonium thiolactate is the ammonium salt of thiolactic acid (i.e., the ammonium salt of 2-sulfanylpropionic acid (formula ii).

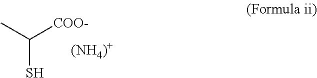

Thiomalic acid is a compound of formula (iii). Thiomalic acid is understood to be D-thiomalic acid, L-thiomalic acid and/or a mixture thereof. Suitable physiologically tolerable salts include the sodium salt, the potassium salt, or the ammonium salt of thiomalic acid.

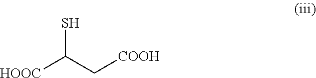

Cysteine is understood to be D-cysteine, L-cysteine and/or a mixture thereof. Alternatively, cysteine is also referred to as 2-amino-3-mercaptopropanoic acid or 2-amino-3-sulfanylpropanoic acid. Suitable physiologically tolerable salts include the sodium salt, the potassium salt, or the ammonium salt of cysteine.

Alternatively, cysteamine is also referred to as 2-aminoethanethiol and has the formula HS—CH₂—CH₂—NH₂. Suitable physiologically tolerable salts of cysteamine include, for example, the hydrochloride, the hydrobromide, the sulfate, or the hemisulfate.

Cystine is a disulfide formed by oxidation of two molecules of the amino acid cysteine. Cystine has CAS number 56-89-3. Also included in the definition of cystine are the (D,D), (D,L), (L,D) and (L,L) isomers. Suitable physiologically tolerable salts include the sodium salt, the potassium salt, or the ammonium salt of cystine.

2-Mercaptoethane sulfonic acid is a compound of formula (IV). Suitable physiologically tolerable salts include, for example, the sodium salt, the potassium salt, or the ammonium salt of 2-mercaptoethane sulfonic acid.

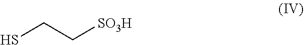

The aforementioned reducing agents are available commercially from various providers (Sigma Aldrich, Merck, Fluka or Alfa Aesar, etc.).

It has been found that certain keratin-reducing compounds are especially compatible with the Bunte salts of formula (I). Most especially preferred are thioglycolic acid, ammonium thioglycolate, thiolactic acid and/or ammonium thiolactate because these reducing agents, when used before, after or together with the Bunte salts of formula (I) within a permanent shape changing process, have led to a particularly great improvement in the permanent wave effect with the best possible protection of the fiber.

In a most especially preferred embodiment, a multicomponent packaging unit as contemplated herein is characterized in that the agent (A) in container (i) contains one or more keratin-reducing compounds from the group of thioglycolic acid, ammonium thioglycolate, thiolactic acid and/or ammonium thiolactate.

Agent (A) contains one or more keratin-reducing compounds preferably in certain quantity ranges to ensure the best possible shaping with the least possible damage. Agent (A) especially preferably contains one or more keratin-reducing compounds in a total amount of from about 1.5% to about 20.0% by weight, preferably from about 3.0% to about 15.0% by weight, more preferably from about 4.0% to about 12.0% by weight and especially preferably from about 4.5% to about 12.5% by weight. All the quantitative amounts here are given in percent by weight (wt %), based on the total weight of the keratin-reducing compounds contained in the agent, which is related to the total weight of the agent (A).

In another most especially preferred embodiment, a multicomponent packaging unit as contemplated herein is characterized in that the agent (A) in container (i) contains—based on the total weight of the agent (A)—one or more keratin-reducing compounds in a total amount of from about 1.5% to about 20.0% by weight, preferably from about 3.0% to about 15.0% by weight, more preferably from about 4.0% to about 12.0% by weight and especially preferably from about 4.5% to about 12.5% by weight.

### Bunte Salts of Formula (I) in Agent (B)

Agent (B), which is present in packaged form in the container (ii), contains as an ingredient essential to the present disclosure at least one Bunte salt of an amino acid, of an oligopeptide, or of a peptide.

The Bunte salt of an amino acid, an oligopeptide or a peptide is described by formula (I)

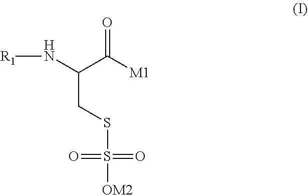

where
R1 stands for a hydrogen atom or for a structural element of the formula (II)

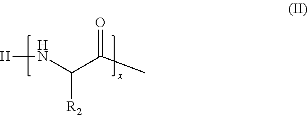

where
x stands for an integer from 1 to about 100,
the radical R2 in each of the structural elements of formula (II) may be selected independently of the previous structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

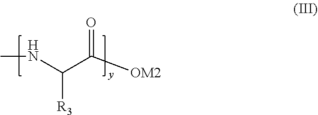

where
y stands for an integer from 1 to about 100,
the radical R3 in each of the structural elements of formula (III) can be selected independently of the aforementioned structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

The R1 radical may stand either for a hydrogen atom or for a structural element of formula (II)

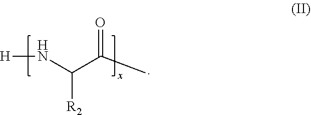

The structural element of formula (II) is also characterized by the repeat index x, where x stands for an integer from 1 to about 100. The repeat index x indicates how many structural elements of formula (II) are contained in the compound of formula (I).

Preferably x stands for an integer from 1 to about 50, more preferably x stands for an integer from 1 to about 20, and most especially preferably x stands for an integer from 1 to about 10.

If x stands for the number 10, for example, then the compound of formula (I) includes 10 structural elements of formula (II).

It is important here that the R2 radical in each one of the structural elements of formula (II) can be selected independently of the preceding structural element of formula (II). For example, if the compounds of formula (I) contain 10 structural units of formula (II), these 10 structural units may be the same but they may also be different.

The R2 radical stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or for a (sulfosulfanyl)methyl group.

If x stands for the number 1, then the compound of formula (I) represents a structural element of formula (II). The linkage of the structural units here is the same as that represented in formula (I-a).

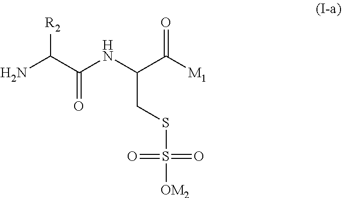

If x stands for the number 2, then the compound of formula (I) contains two structural elements of formula (II). The linkage of the structural units here is the same as that represented in formula (I-b).

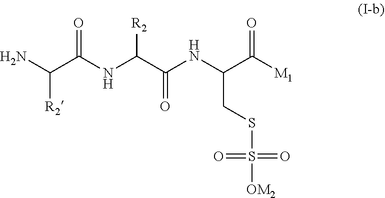

As described previously, each R2 radical here can be selected independently of the other R2 radicals. The R2 radicals that can be selected, independently of one another, are characterized by R2 and R2′ in formula (I-b).

If x stands for the number 3, then the compound of formula (I) contains three structural elements of formula (II). The linkage of the structural units here is the same as that represented in formula (I-c).

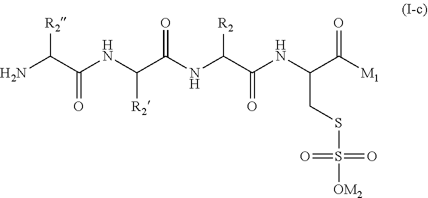

As described previously, each of the R2 radicals here can be selected independently of the other R2 radicals. The R2 radicals that can be selected, independently of one another, are characterized by R2, R2′ and R2″ in formula (I-c).

If x stands for the number 4, then the compound of formula (I) contains four structural elements of formula (II). The linkage of the structural units here is the same as that represented in formula (I-d).

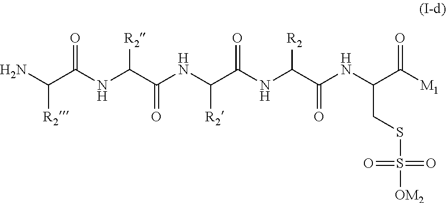

As described above, each R2 radical here can be selected independently of the other R2 radicals. The R2 radicals that can be selected, independently of one another, are characterized by R2, R2′, R2″ and R2″ in formula (I-d).

If x stands for the number 5, then the compound of formula (I) contains five structural elements of formula (II). The linkage of the structural units here is the same as that represented in formula (I-e).

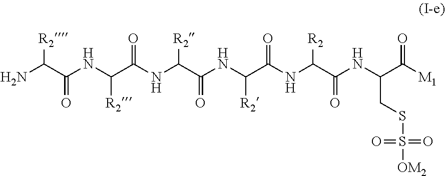

As described above, each R2 radical here can be selected independently of the other R2 radicals. The R2 radicals that can be selected, independently of one another, are characterized by R2, R2′, R2″, R2′″ and R2″″ in formula (I-e).

If x stands for the number 6, the compound of formula (I) contains six structural elements of formula (II). The linkage of the structural units here is the same as that represented in formula (I-f).

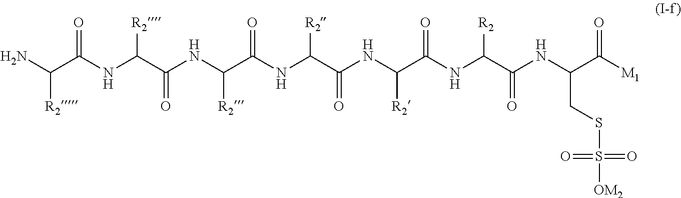

As described previously, each R2 radical here can be selected independently of the other R2 radicals. The R2 radicals that can be selected, independently of one another, are characterized by R2, R2′, R2″, R2′″, R″″ and R″″′ in formula (I-f).

The linkage of all other x structural units is done similarly.

The structural element of formula (II) is thus an amino acid, which has a peptide link via its amino function and/or its acid function within the compound of formula (I). If the amino acid is cysteine, this may also be present in the form of a Bunte salt.

If the R2 radical stands for a hydrogen atom, then the structural element of formula (II) is based on the amino acid glycine.

If the R2 radical stands for a methyl group, then the structural element of formula (II) is based on the amino acid alanine.

If the R2 radical stands for an isopropyl group (i.e., a (H₃C)₂CH— group), then the structural element of formula (II) is based on the amino acid valine.

If the R2 radical stands for a 2-methylpropyl group (i.e., a (H₃C)₂CH—CH₂— group), then the structural element of formula (II) is based on the amino acid leucine.

If the R2 radical stands for a 1-methylpropyl group (i.e., a H₃C—CH₂—CH(CH₃)— group), then the structural element of formula (II) is based on the amino acid isoleucine.

If the R2 radical stands for a benzyl group (i.e., a C₆H₅—CH₂— group), then the structural element of formula (II) is based on the amino acid phenylalanine.

If the R2 radical stands for a 4-hydroxybenzyl group (i.e., a 4-OH—C₆H₅—CH₂— group), then the structural element of formula (II) is based on the amino acid tyrosine.

If the R2 radical stands for a hydroxymethyl group (i.e., a HO—CH₂— group), then the structural element of formula (II) is based on the amino acid serine.

If the R2 radical stands for a 1-hydroxyethyl group (i.e., a H₃C—CH(OH)— group), then the structural element of formula (II) is based on the amino acid threonine.

If the R2 radical stands for a 4-aminobutyl group (i.e., a H₂N—CH₂—CH₂—CH₂—CH₂— group), then the structural element of formula (II) is based on the amino acid lysine.

If the R2 radical stands for a 3-carbamimidamidopropyl group (i.e., a H₂N—C(NH)—NH—CH₂—CH₂—CH₂— group), then the structural element of formula (II) is based on the amino acid arginine.

If the R2 radical stands for a 2-carboxyethyl group (i.e., a HOOC—CH₂—CH₂— group), then the structural element of formula (II) is based on the amino acid glutamic acid.

If the R2 radical stands for a carboxymethyl group (i.e., a HOOC—CH₂— group), then the structural element of formula (II) is based on the amino acid aspartic acid.

If the R2 radical stands for a 2-carbamoylethyl group (i.e., a H₂N—C(O)—CH₂—CH₂— group), then the structural element of formula (II) is based on the amino acid glutamine.

If the R2 radical stands for a carbamoylmethyl group (i.e., a H₂N—C(O)—CH₂— group), then the structural element of formula (II) is based on the amino acid asparagine.

If the R2 radical stands for a sulfanylmethyl group (i.e., a HS—CH₂— group), then the structural element of formula (II) is based on the amino acid cysteine.

If the R2 radical stands for a 2-(methylsulfanyl)ethyl group (i.e., a H₃C—S—CH₂—CH₂—group), then the structural element of formula (II) is based on the amino acid methionine.

If the R2 radical stands for a 1H-imidazol-4-ylmethyl group, then the structural element of formula (II) is based on the amino acid histidine.

If the R2 radical stands for a 1H-indol-3-ylmethyl group, then the structural element of formula (II) is based on the amino acid tryptophan.

Finally, the R2 radical may also stand for a (sulfosulfanyl)methyl group, so this is a Bunte salt structure of the formula HO—S(O₂)—S—CH₂—.

Depending on the pH of the hair shaping agent, the Bunte salt structure of the formula HO—S(O₂)—S—CH₂— may also be present in its deprotonated form.

In another particularly preferred embodiment, a multicomponent packaging unit as contemplated herein is characterized in that
the agent (B) in container (ii) contains at least one compound of general formula (I-a)

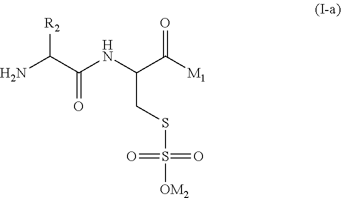

where
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

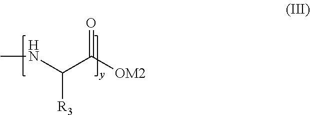

where
y stands for an integer from 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group, and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that
the agent (B) in container (ii) contains at least one compound of general formula (I-b)

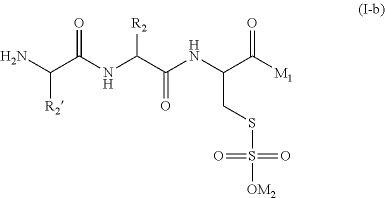

where
R2 and R2′, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

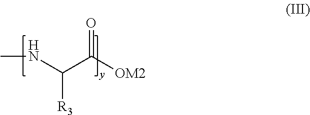

where
y stands for an integer from about 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that
the agent (B) in container (ii) contains at least one compound of the general formula (I-c)

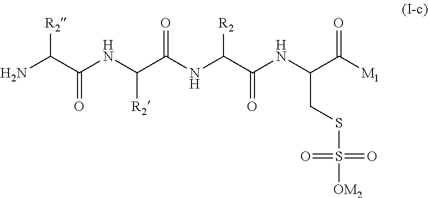

where
R2, R2′ and R2″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

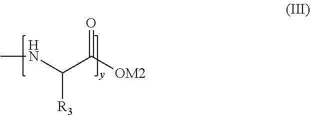

where
y stands for an integer from 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺ and
In another particularly preferred embodiment, a multicomponent packaging unit as contemplated herein is characterized in that
the agent (B) in container (ii) contains at least one compound of the general formula (I-d)

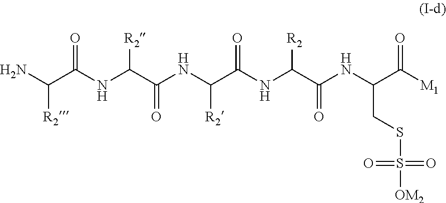

where
R2, R2′, R2″ and R2′″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

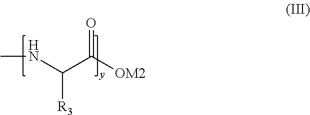

where
y stands for an integer from about 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that
the agent (B) in container (ii) contains at least one compound of the general formula (I-e)

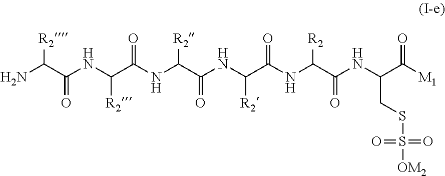

where
R2, R2′, R2″, R2′″ and R2″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

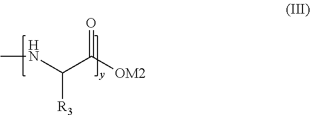

where
y stands for an integer from 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that
the agent (B) in container (ii) contains at least one compound of the general formula (I-f)

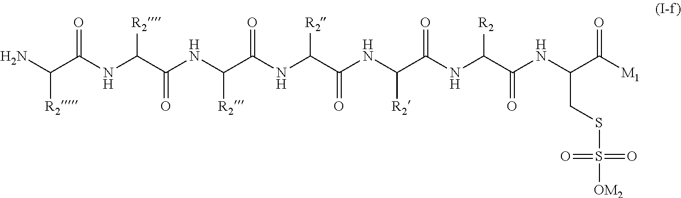

where
R2, R2′, R2″, R2′″, R2″″ and R″″′, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)-ethyl group, a 1H-imidazol-4-ylmethyl group, a 1H-indol-3-ylmethyl group or a (sulfosulfanyl)-methyl group,
M1 stands for the —OM2 group or for a structural element of formula (III):

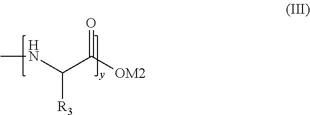

where
y stands for an integer from about 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

Within the compound of formula (I), M1 stands for the —OM2 group or for a structural element of formula (III).

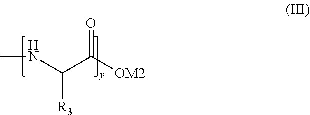

The structural element of formula (III)—exactly like the structural element of formula (II)—is characterized by a repeat index. The repeat index y stands for an integer from 1 to about 100. The repeat index y indicates how many structural elements of formula (III) are contained in the compound of formula (I).

Preferably y stands for an integer from about 1 to about 50, more preferably y stands for an integer from 1 to about 20 and most especially preferably y stands for an integer from 1 to about 10.

For example, if y stands for the integer 10, then the compound of formula (I) contains 10 structural elements of formula (III).

It is important here that the R3 radical in each of the structural elements of formula (III) can be selected independently of the preceding structural element of formula (III). If the compounds of formula (I) contain 10 structural units of formula (III), for example, then these 10 structural units may be the same or may be different.

The R3 radical stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group.

Therefore the structural element of formula (III) is also an amino acid having a peptide link via its amino function and/or its acid function within the compound of formula (I). If the amino acid is cysteine, it may also be present in the form of a Bunte salt.

If y stands for the number 1, then the compound of formula (I) contains a structural element of formula (III). The linkage of the structural units here is the same as that in formula (I-I).

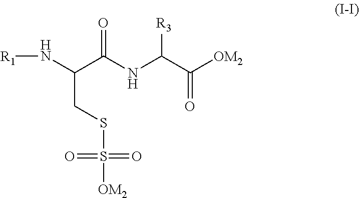

If y stands for the number 2, then the compound of formula (I) contains two structural elements of formula (III). The linkage of the structural units here is represented the same as that in formula (I-II).

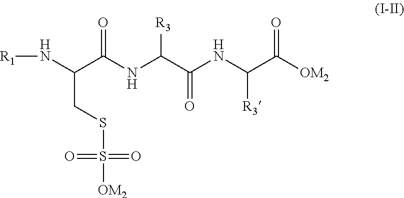

As described previously, each of the R3 radicals here can be selected independently of the other R3 radical. The R3 radicals that can be selected, independently of one another, are characterized by R3 and R3′ in formula (I-II).

If y stands for the number 3, then the compound of formula (I) contains three structural elements of formula (III). The linkage of the structural units here is represented the same as that in formula (I-III).

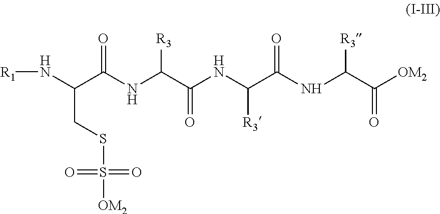

As described previously, each of the R3 radicals here can be selected independently of the other R3 radical. The R3 radicals that can be selected, independently of one another, are characterized by R3, R3′ and R3″ in formula (I-III).

If y stands for the number 4, then the compound of formula (I) contains four structural elements of formula (III). The linkage of the structural units here is represented the same as that in formula (I-IV).

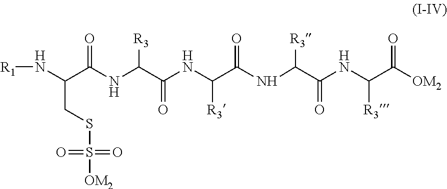

As described previously, each of the R3 radicals here can be selected independently of the other R3 radical. The R3 radicals that can be selected, independently of one another, are characterized by R3, R3′, R3″ and R3″ in formula (I-IV).

If y stands for the number 5, then the compound of formula (I) contains five structural elements of formula (II). The linkage of the structural units here is represented the same as that in formula (I-V).

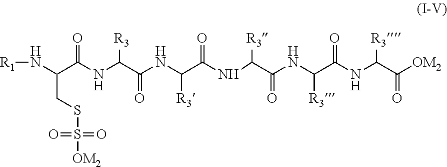

As described previously, each of the R3 radicals here can be selected independently of the other R3 radical. The R3 radicals that can be selected, independently of one another, are characterized by R3, R3′, R3″, R3″ and R3″″ in formula (I-V).

If y stands for the number 6, then the compound of formula (I) contains six structural elements of formula (II). The linkage of the structural units here is represented the same as that in formula (I-VI).

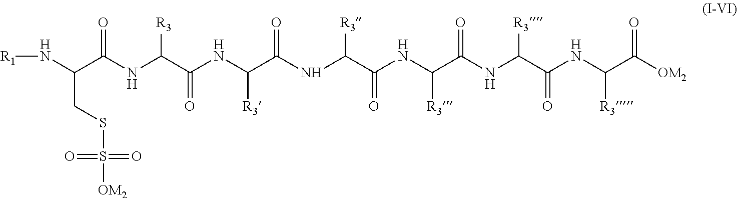

As described previously, each of the R3 radicals here can be selected independently of the other R3 radical. The R3 radicals that can be selected, independently of one another, are characterized by R3, R3′, R3″, R3″, R3″″ and R3″″′ in formula (I-VI).

In another particularly preferred embodiment, a multicomponent packaging unit as contemplated herein is characterized in that
the agent (B) in container (ii) contains at least one compound of general formula (I-I)

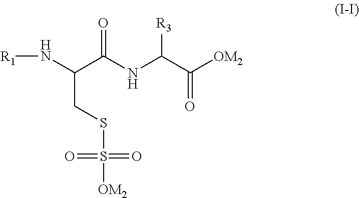

where
R1 stands for a hydrogen atom or a structural element of formula (II)

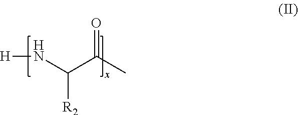

where
x stands for an integer from 1 to about 10,
the R2 radical in each of the structural element of formula (II) can be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that
the agent (B) in container (ii) contains at least one compound of the general formula (I-II)

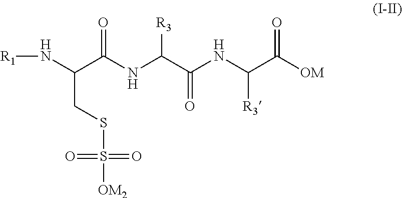

where
R1 stands for a hydrogen atom or a structural element of formula (II)

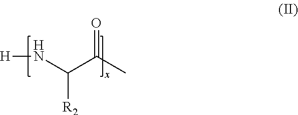

where
x stands for an integer from 1 to about 10,
the R2 radical in each of the structural element of formula (II) can be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
R3 and R3′, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that
the agent (B) in container (ii) contains at least one compound of the general formula (I-III)

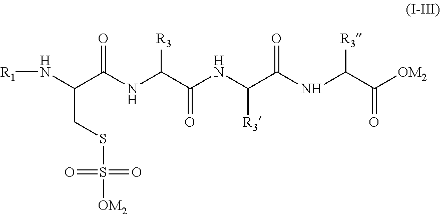

where
R1 stands for a hydrogen atom or a structural element of formula (II)

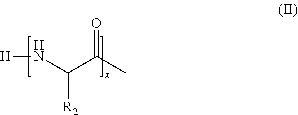

where
x stands for an integer from about 1 to about 10,
the R2 radical in each of the structural element of formula (II) can be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
R3, R3′ and R3″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that
the agent (B) in container (ii) contains at least one compound of the general formula (I-IV)

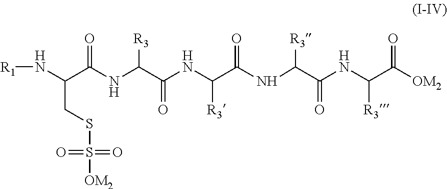

where
R1 stands for a hydrogen atom or a structural element of formula (II)

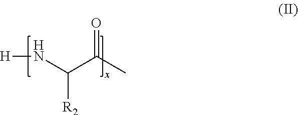

where
x stands for an integer from 1 to about 10,
the R2 radical in each of the structural element of formula (II) can be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
R3, R3′, R3″ and R3′″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

n another particularly preferred embodiment, a multicomponent packaging unit as contemplated herein is characterized in that
the agent (B) in container (ii) contains at least one compound of the general formula (I-V)

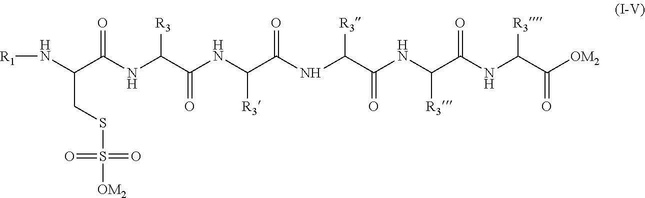

where
R1 stands for a hydrogen atom or a structural element of formula (II)

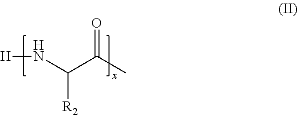

where
x stands for an integer from 1 to about 10,
the R2 radical in each of the structural element of formula (II) can be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
R3, R3′, R3″, R′″ and R3″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that
the agent (B) in container (ii) contains at least one compound of the general formula (I-VI)

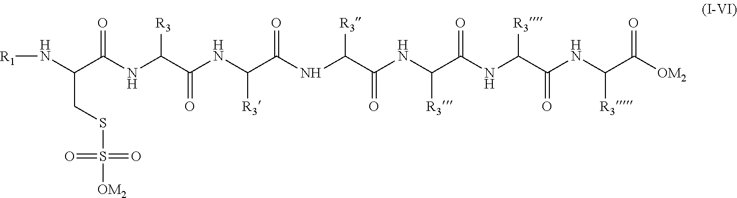

where
R1 stands for a hydrogen atom or a structural element of formula (II)

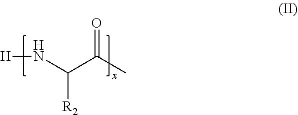

where
x stands for an integer from 1 to about 10,
the R2 radical in each of the structural element of formula (II) can be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
R3, R3′, R3″, R′″, R″″ and R3″″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

If the R3 radical stands for a hydrogen atom, then the structural element of formula (II) is based on the amino acid glycine.

If the R3 radical stands for a methyl group, then the structural element of formula (II) is based on the amino acid alanine.

If the R3 radical stands for an isopropyl group (i.e., a (H₃C)₂CH— group), then the structural element of formula (II) is based on the amino acid valine.

If the R3 radical stands for a 2-methylpropyl group (i.e., a (H₃C)₂CH—CH₂— group), then the structural element of formula (II) is based on the amino acid leucine.

If the R3 radical stands for a 1-methylpropyl group (i.e., a H₃C—CH₂—CH(CH₃)— group), then the structural element of formula (II) is based on the amino acid isoleucine.

If the R3 radical stands for a benzyl group (i.e., a C₆H₅—CH₂— group), then the structural element of formula (II) is based on the amino acid phenylalanine.

If the R3 radical stands for a 4-hydroxybenzyl group (i.e., a 4-OH—C₆H₅—CH₂— group), then the structural element of formula (II) is based on the amino acid tyrosine.

If the R3 radical stands for a hydroxymethyl group (i.e., a HO—CH₂— group), then the structural element of formula (II) is based on the amino acid serine.

If the R3 radical stands for a 1-hydroxyethyl group (i.e., a H₃C—CH(OH)— group), then the structural element of formula (II) is based on the amino acid threonine.

If the R3 radical stands for a 4-aminobutyl group (i.e., a H₂N—CH₂—CH₂—CH₂—CH₂— group), then the structural element of formula (II) is based on the amino acid lysine.

If the R3 radical stands for a 3-carbamimidamidopropyl group (i.e., a H₂N—C(NH)—NH—CH₂—CH₂—CH₂— group), then the structural element of formula (II) is based on the amino acid arginine.

If the R3 radical stands for a 2-carboxyethyl group (i.e., a HOOC—CH₂—CH₂— group), then the structural element of formula (II) is based on the amino acid glutamic acid.

If the R3 radical stands for a carboxymethyl group (i.e., a HOOC—CH₂— group), then the structural element of formula (II) is based on the amino acid aspartic acid.

If the R3 radical stands for a 2-carbamoylethyl group (i.e., a H₂N—C(O)—CH₂—CH₂— group), then the structural element of formula (II) is based on the amino acid glutamine.

If the R3 radical stands for a carbamoylmethyl group (i.e., a H₂N—C(O)—CH₂— group), then the structural element of formula (II) is based on the amino acid asparagine.

If the R3 radical stands for a sulfanylmethyl group (i.e., a HS—CH₂— group), then the structural element of formula (II) is based on the amino acid cysteine.

If the R3 radical stands for a 2-(methylsulfanyl)ethyl group (i.e., a H₃C—S—CH₂—CH₂—group), then the structural element of formula (II) is based on the amino acid methionine.

If the R3 radical stands for a 1H-imidazol-4-ylmethyl group, then the structural element of formula (II) is based on the amino acid histidine.

If the R3 radical stands for a 1H-indol-3-ylmethyl group, then the structural element of formula (II) is based on the amino acid tryptophan.

Finally the R3 radical may also stand for a (sulfosulfanyl)methyl group, so this is a Bunte salt structure of the formula HO—S(O₂)—S—CH₂—.

Depending on the pH of the hair shaping agent, the Bunte salt structure of the formula HO—S(O₂)—S—CH₂— may also be present in its deprotonated form.

The M2 radical stands for a hydrogen atom, one equivalent of a monovalent or polyvalent cations or an ammonium ion (NH₄)⁺.

In particular the cations of sodium and potassium (Na⁺ and/or K⁺) or magnesium or calcium (½Mg²⁺ or ½Ca²⁺) can be mentioned as preferred equivalents of a monovalent cation.

If M2 stands for a hydrogen atom, then the —OM2 group is the —OH group. If M2 stands for sodium cation, then the —OM2 group is the —ONa group. If M2 stands for potassium cation, then the —OM2 group is the —OK group. If M2 stands for an ammonium ion, then the —OM2 group is the —O(NH₄) group.

The inventive compounds of formula (I) are either the Bunte salt of the amino acid cysteine, the Bunte salts of oligopeptides or the Bunte salts of peptides.

If the R1 radical stands for a hydrogen atom and the M1 radical stands for an —OM2 group, then the compound of formula (I) is the Bunte salt of the amino acid cysteine. In this case, the compound of formula (I) is the compound of formula (IA)

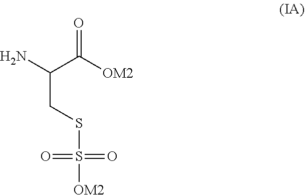

where M2 is again defined as described above.

If the compound of formula (IA) is in the form of its free acid, then it is 2-amino-3-(sulfosulfanyl)propanoic acid. This sub stance is commercially available.

It has been found that use of the compound of formula (IA) in hair shaping agents results in a particularly effective reduction in hair damage even when used in particularly small amounts, and this effect persists even after repeated shampooing. Therefore the use of compounds of formula (IA) in hair shaping agents is most especially preferred.

In a most especially preferred embodiment, a multicomponent packaging unit as contemplated herein according to claims 1 through 4 is characterized in that the agent (B) in container (ii) contains at least one compound of formula (I), where

R1 stands for a hydrogen atom and
M1 stands for an —OM2 group.

If a compound of formula (IA) is used, it is preferable to use this specific compound. However, if the Bunte salts of oligopeptides are used as the compounds of formula (I), then the inventive hair shaping agent may also contain a plurality of compounds of formula (I) as a mixture of various oligopeptides. These oligopeptides are defined by their average molecular weight. The average molecular weight Mw of the at least one oligopeptide of formula (I) can be determined, for example, by gel permeation chromatography (GPC) with polystyrene as the internal standard according to DIN 55672-3.

Depending on how many structural elements of formulas (II) and/or (III) are contained in the compound of formula (I) and depending on the type of these amino acids, the molecular weight of the compound of formula (I) used as contemplated herein can vary. It is particularly preferred as contemplated herein if the compound of formula (I) is an oligopeptide having a molecular weight Mw of 200 to 2000 Da, preferably of from about 250 to about 1500 Da, preferably of from about 300 to about 1200 Da, in particular of from about 400 to about 800 Da.

The term “oligopeptide” is understood within the scope of the present disclosure to refer to condensation products of amino acids, which have the molecular weights referenced above.

In a most especially preferred embodiment, a multicomponent packaging unit as contemplated herein is characterized in that the agent (B) in container (ii) contains at least one compound of formula (I) having a molecular weight Mw of from about 200 to about 2000 Da, preferably of from about 250 to about 1500 Da, especially of from about 300 to about 1200 Da, in particular of from about 400 to about 800 Da.

If a mixture of oligomers is used in the inventive hair shaping agent, then these mixtures can be defined by their average molecular weight.

In a most especially preferred embodiment, a multicomponent packaging unit as contemplated herein is characterized in that the agent (B) in container (ii) contains compounds of formula (I) having an average molecular Mw of from about 200 to about 2000 Da, preferably of from about 250 to about 1500 Da, especially of from about 300 to about 1200 Da, in particular of from about 400 to about 800 Da,

In addition it has been found that the repair effect of the compounds of formula (I) also depends on the repeating indices x and y. As already described above, it is most especially preferred if x stands for an integer from about 1 to about 10 and if y stands for an integer from about 1 to about 10.

In another most especially preferred embodiment, a multicomponent packaging unit as contemplated herein is characterized in that the agent (B) in container (ii) contains at least one compound of formula (I), wherein

R1 stands for a structural element of formula (II), and
M1 stands for a structural element of formula (III), and
x stands for an integer from 1 to about 10 and
y stands for an integer from 1 to about 10.

In addition to the molecular weight of the compound of formula (I), the amount of Bunte salt units contained in the compound of formula (I) also has a significant influence on the efficacy of the “repair effect” of the compounds.

Compounds having at least one Bunte salt unit—such as that present in the compound of formula (Ia)—are very effective, in particular when they are used as a monomeric compound. Oligopeptides having at least Bunte salt unit are particularly effective if they have a low molecular weight of up to about 1200, in particular about 800 Dalton.

When using oligopeptides, however, it is most especially advantageous if the compound of formula (I) has at least two, preferably at least three Bunte salt units.

In another most especially preferred embodiment, a multicomponent packaging unit as contemplated herein is characterized in that the agent (B) in container (ii) contains at least one compound of formula (I), wherein

R1 stands for a structural element of formula (II), and
the R2 radical in at least one structural unit of formula (II) stands for a (sulfosulfanyl)methyl group (i.e., a HO—S(O)₂)—S—CH₂— group).

In another particularly preferred embodiment, a multicomponent packaging unit as contemplated herein is characterized in that the agent (B) in container (ii) contains at least one compound of the formula (I-a′)

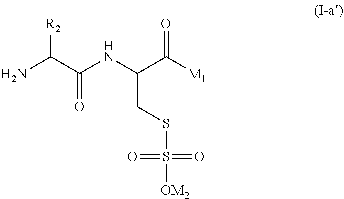

where
R2 stands for a (sulfosulfanyl)methyl group (i.e., a HO—S(O₂)—S—CH₂— group) and
M1 stands for the —OM2 group or for a structural element of formula (III):

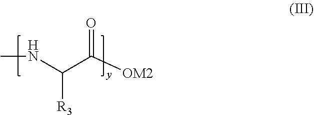

where
y stands for an integer from 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-b′)

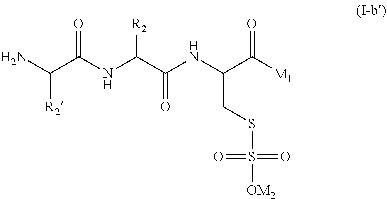

where
one of the radicals of R2 and R2′ stands for a (sulfosulfanyl)methyl group (i.e., a HO—S(O₂)—S—CH₂— group) and
the other one of the R2 and R2′ radicals stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

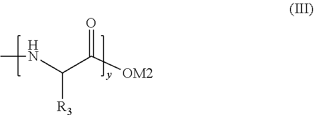

where
y stands for an integer from about 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-c′)

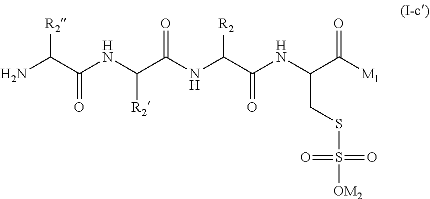

where
one of the radicals of R2, R2′ and R2″ stands for a (sulfosulfanyl)methyl group (i.e., an HO S(O₂) S CH₂— group) and
the other two radicals of R2, R2′ and R2″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

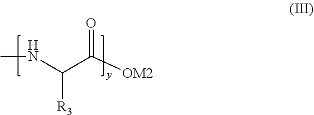

where
y stands for an integer from 1 to about 10,
the R3 radical in each of the structural elements of formula (III) can be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-d′)

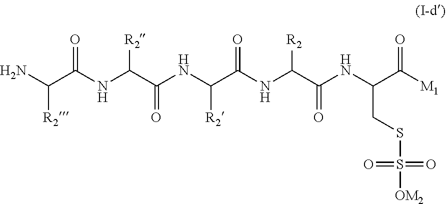

where
one of the radicals of R2, R2′, R2″ and R2′″ stands for a (sulfosulfanyl)methyl group (i.e., an HO—S(O₂)—S—CH₂— group) and
the other three radicals of R2, R2′, R2″ and R2″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

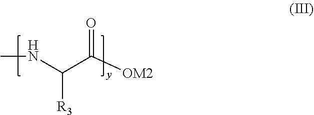

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

n another particularly preferred embodiment, a multicomponent packaging unit as contemplated herein is characterized in that the agent (B) in container (ii) contains at least one compound of the formula (I-e′)

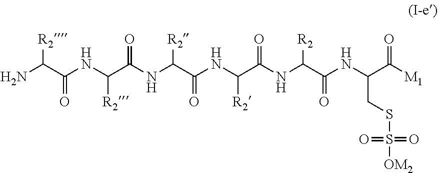

where
one of the radicals of R2, R2′, R2″, R2″ and R2″″ stands for a (sulfosulfanyl)methyl group (i.e., an HO—S(O₂)—S—CH₂— group) and
the other four radicals of R2, R2′, R2″, R2′″ and R2″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

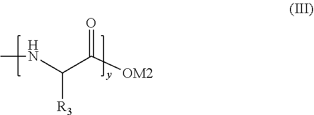

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-f′)

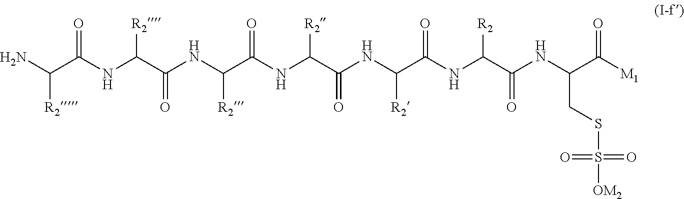

where
one of the radicals of R2, R2′, R2″, R2′″, R2″″ and R2″″′ stands for a (sulfosulfanyl)methyl group (i.e., an HO—S(O₂)—S—CH₂— group) and the other five radicals of R2, R2′, R2″, R2′″, R2″″ and R2″″′, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

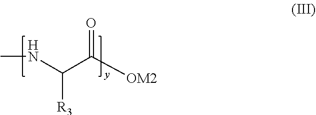

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another most especially preferred embodiment, a multicomponent packaging unit as contemplated herein is characterized in that the agent (B) in container (ii) contains at least one compound of formula (I) wherein

R1 stands for a structural element of formula (II) and
x stands for an integer of at least 3 and
the R2 radical in at least one structural element of formula (II) stands for a 2-carboxyethyl group. In another particularly preferred embodiment, a multicomponent packaging unit as contemplated herein is characterized in that the agent (B) in container (ii) contains at least one compound of the formula (I-a″)

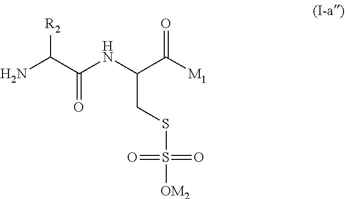

where
R2 stands for a 2-carboxyethyl group (i.e., an HOOC—CH₂—CH₂— group) and
M1 stands for the —OM2 group or for a structural element of formula (III):

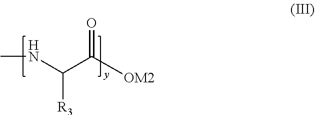

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-b″)

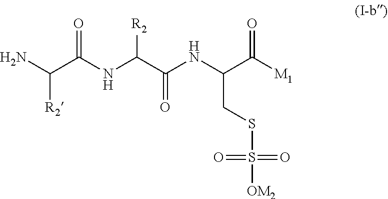

where
one of the radicals of R2 and R2′ stands for a 2-carboxyethyl group (i.e., an HOOC—CH₂—CH₂—group) and
the other one of the R2 and R2′ radicals stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

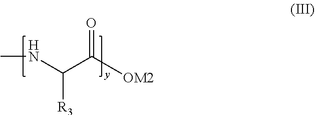

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-c″)

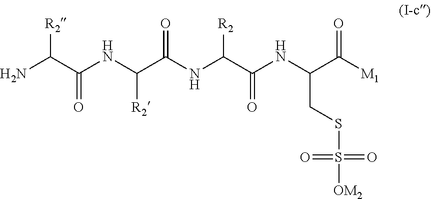

where
one of the radicals of R2, R2′ and R2″ stands for a 2-carboxyethyl group (i.e., an HOOC—CH₂—CH₂— group) and
the other radicals of R2, R2′ and R2″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

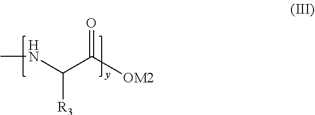

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-d″)

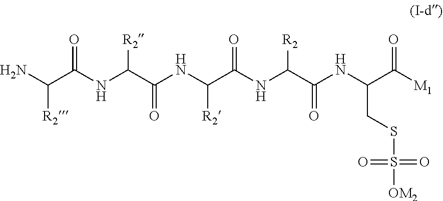

where
one of the radicals of R2, R2′, R2″ and R2′″ stands for a 2-carboxyethyl group (i.e., an HOOC—CH₂—CH₂— group) and
the other radicals of R2, R2′, R2″ and R2″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

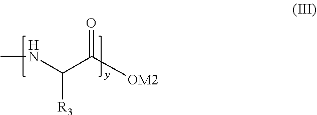

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-e″)

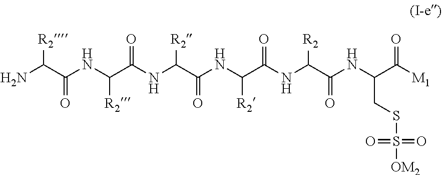

where
one of the radicals of R2, R2′, R2″, R2″ and R2″″ stands for a 2-carboxyethyl group (i.e., an HOOC—CH₂—CH₂— group) and
the other radicals of R2, R2′, R″, R′″ and R2″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

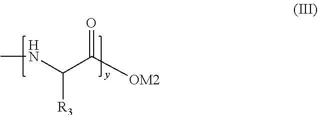

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I-f″)

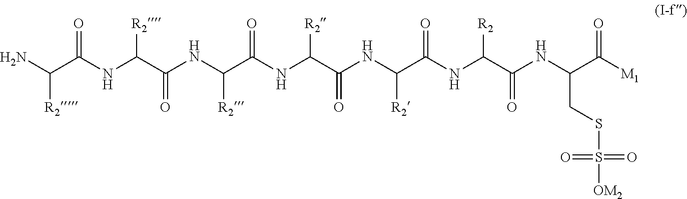

where
at least one of the radicals R2, R2′, R2″, R2″, R2″″ and R2″″ stands for a 2-carboxyethyl group (i.e., an HOOC—CH₂—CH₂— group), and
the other five radicals of R2, R2′, R2″, R2′″, R2″″ and R2″″′, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

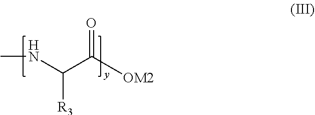

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another most especially preferred embodiment, a multicomponent packaging unit as contemplated herein is characterized in that the agent (B) in container (ii) contains at least one compound of formula (I) wherein

R1 stands for a structural element of formula (II), and
x stands for an integer of at least 3, and
the R2 radical in at least three structural elements of formula (II) stands for a 2-carboxyethyl group.

In another particularly preferred embodiment, a multicomponent packaging unit as contemplated herein is characterized in that the agent (B) in container (ii) contains at least one compound of formula (I):

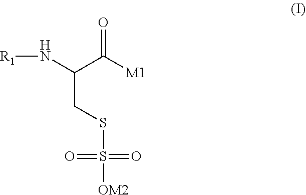

R1 stands for a structural element of formula (II)

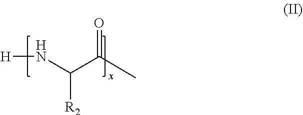

where
x stands for an integer from 3 to about 10,
the R2 radical in each of the structural elements of formula (II) can be selected independently of the preceding structural element of formula (II),
the R2 radical in at least three structural elements of formula (II) stands for a 2-carboxyethyl group, the other R2 radicals stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III):

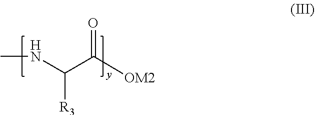

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another most especially preferred embodiment, a multicomponent packaging unit as contemplated herein is characterized in that the agent (B) in container (ii) contains at least one compound of formula (I) wherein

M1 stands for a structural element of formula (III), and
the R3 radical in at least one structural element of formula (III) stands for a (sulfosulfanyl)methyl group (i.e., an HO—S(O₂)—S—CH₂— group).

In another particularly preferred embodiment, a multicomponent packaging unit as contemplated herein is characterized in that agent (B) in container (ii) contains at least one compound of formula (I-I′):

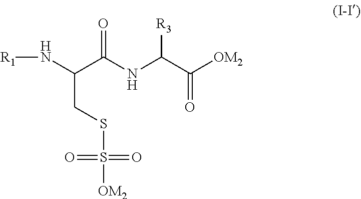

where
R1 stands for a hydrogen atom or for a structural element of formula (II):

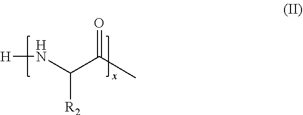

where
x stands for an integer from 1 to about 10,
the R2 radical in each of the structural elements of formula (II) can each be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
R3 stands for a (sulfosulfanyl)methyl group (i.e., an HO—S(O₂)—S—CH₂— group), and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of formula (I-II′):

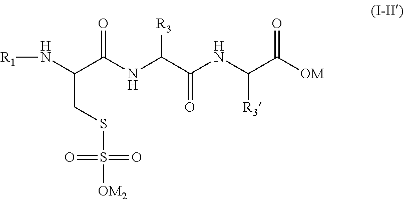

where
R1 stands for a hydrogen atom or for a structural element of formula (II):

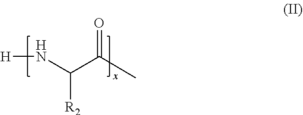

where
x stands for an integer from 1 to about 10,
the R2 radical in each of the structural elements of formula (II) can each be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3 and R3′ stands for a (sulfosulfanyl)methyl group (i.e., an HO—S(O₂)—S—CH₂— group), and
the other radicals of R3 and R3′, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group, and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of formula (I-III′):

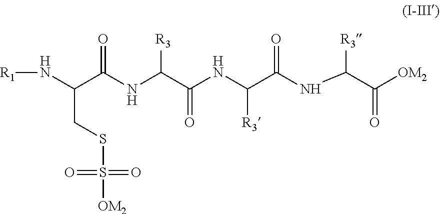

where
R1 stands for a hydrogen atom or for a structural element of formula (II):

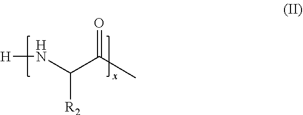

where
x stands for an integer from 1 to about 10,
the R2 radical in each of the structural elements of formula (II) can each be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3, R3′ and R3″ stands for a (sulfosulfanyl)methyl group (i.e., an HO—S(O₂)—S—CH₂— group), and
the other radicals of R3, R3′ and R3″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)-ethyl group, a 1H-imidazol-4-ylmethyl group, a 1H-indol-3-ylmethyl group or a (sulfosulfanyl)methyl group, and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of formula (I-IV′):

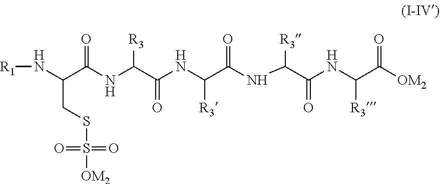

where
R1 stands for a hydrogen atom or for a structural element of formula (II):

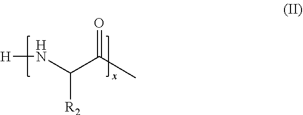

where
x stands for an integer from 1 to about 10,
the R2 radical in each of the structural elements of formula (II) can each be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3, R3′, R3″ and R3′″ stands for a (sulfosulfanyl)methyl group (i.e., an HO—S(O₂)—S—CH₂— group), and
the other radicals of R3, R3′, R3″ and R3″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group, and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of formula (I-V′):

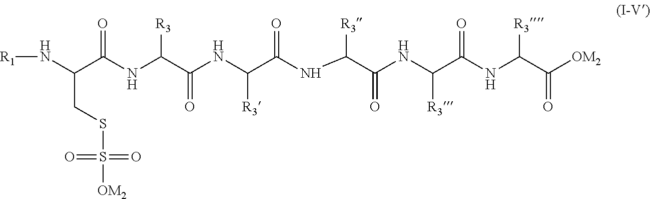

where
R1 stands for a hydrogen atom or for a structural element of formula (II):

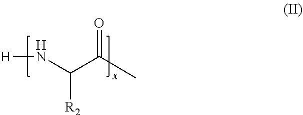

where
x stands for an integer from 1 to about 10,
the R2 radical in each of the structural elements of formula (II) can each be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3, R3′, R3″, R3″ and R3″″ stands for a (sulfosulfanyl)methyl group (i.e., an HO—S(O₂)—S—CH₂— group), and
the other radicals of R3, R3′, R3″, R3″ and R3″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group, and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of formula (I-VI′):

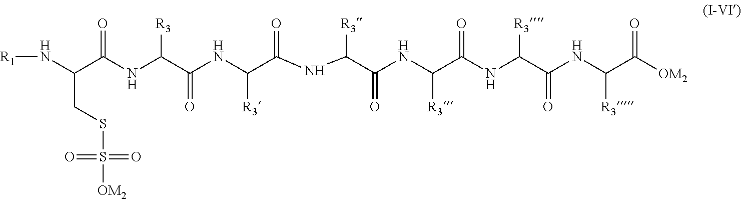

where
R1 stands for a hydrogen atom or for a structural element of formula (II):

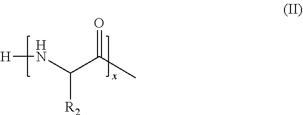

where
x stands for an integer from 1 to about 10,
the R2 radical in each of the structural elements of formula (II) can each be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3, R3′, R3″, R3′″, R3″″ and R3″″′ stands for a (sulfosulfanyl)methyl group (i.e., an HO—S(O₂)—S—CH₂— group), and
the other radicals of R3, R3′, R3″, R3′″, R3″″ and R3″″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another particularly preferred embodiment, a multicomponent packaging unit as contemplated herein is characterized in that the agent (B) in container (ii) contains at least one compound of the formula (I)

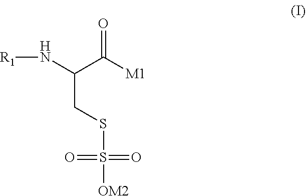

where
R1 stands for a structural element of formula (II)

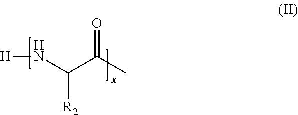

where
x stands for an integer from 3 to about 10,
the R2 radical in each of the structural elements of formula (II) may be selected independently of the preceding structural element of formula (II),
the R2 radical in at least three structural elements of formula (II) stands for a 2-carboxyethyl group (i.e., an HOOC—CH₂—CH₂— group) and
the R2 in the remaining structural elements of formula (II) stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for a structural element of formula (III):

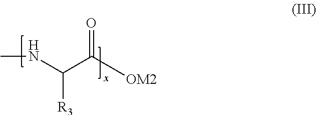

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another most especially preferred embodiment, a multicomponent packaging unit as contemplated herein for permanent shaping of keratinic fibers is characterized in that the agent (B) in container (ii) contains at least one compound of formula (I) wherein

M1 stands for a structural element of formula (III), and
y stands for an integer from about 1 to about 10, and
the R3 radical in at least one structural element of formula (III) stands for a 2-carboxyethyl group (i.e., an HOOC—CH₂—CH₂— group).

In another most especially preferred embodiment, a multicomponent packaging unit as contemplated herein for permanent shaping of keratinic fibers is characterized in that the agent (B) in container (ii) contains at least one compound of formula (II″) [sic; (I-I″)] wherein

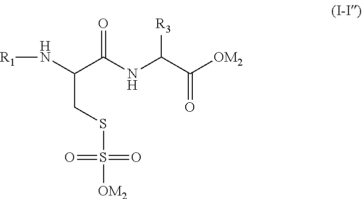

where
R1 stands for a hydrogen atom or for a structural element of formula (II)

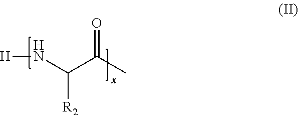

where
x stands for an integer from 1 to about 10
the R2 radical in each of the structural elements of formula (II) may be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
R3 stands for a 2-carboxyethyl group (i.e., an HOOC—CH₂—CH₂— group) and
M2 stands for a hydrogen atom, one equivalent of a monovalent or polyvalent cation or an ammonium ion (NH₄)⁺.

In another most especially preferred embodiment, a multicomponent packaging unit as contemplated herein for permanent shaping of keratinic fibers is characterized in that the agent (B) in container (ii) contains at least one compound of formula (I-II′) wherein

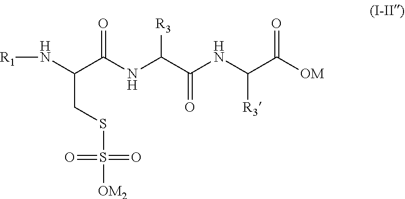

where
R1 stands for a hydrogen atom or for a structural element of formula (II)

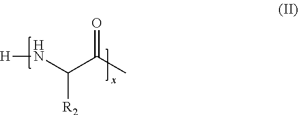

where
x stands for an integer from 1 to about 10,
the R2 radical in each of the structural elements of formula (II) may be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3 and R3′ stands for a 2-carboxyethyl group (i.e., an HOOC—CH₂—CH₂— group) and
the other radicals of R3 and R3′, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another most especially preferred embodiment, a multicomponent packaging unit as contemplated herein for permanent shaping of keratinic fibers is characterized in that the agent (B) in container (ii) contains at least one compound of formula (I-III′) wherein

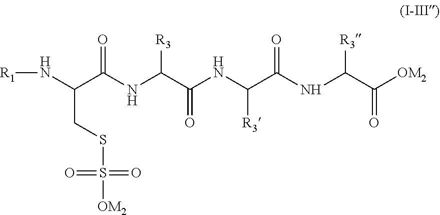

where
R1 stands for a hydrogen atom or for a structural element of formula (II)

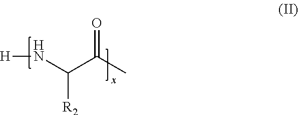

where
x stands for an integer from 1 to about 10,
the R2 radical in each of the structural elements of formula (II) may be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3, R3′ and R3″ stands for a 2-carboxyethyl group (i.e., an HOOC—CH₂—CH₂— group) and
the other radicals of R3, R3′ and R3″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another most especially preferred embodiment, a multicomponent packaging unit as contemplated herein for permanent shaping of keratinic fibers is characterized in that the agent (B) in container (ii) contains at least one compound of formula (I-IV″) wherein

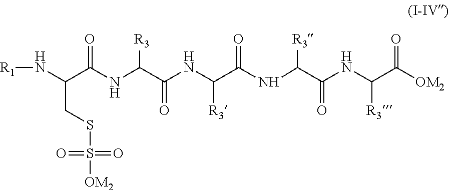

where
R1 stands for a hydrogen atom or for a structural element of formula (II)

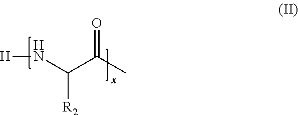

where
x stands for an integer from 1 to about 10,
the R2 radical in each of the structural elements of formula (II) may be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3, R3′, R3″ and R3′″ stands for a 2-carboxyethyl group (i.e., an HOOC—CH₂—CH₂— group) and
the other radicals of R3, R3′, R3″ and R3″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another most especially preferred embodiment, a multicomponent packaging unit as contemplated herein for permanent shaping of keratinic fibers is characterized in that the agent (B) in container (ii) contains at least one compound of formula (I-V″) wherein

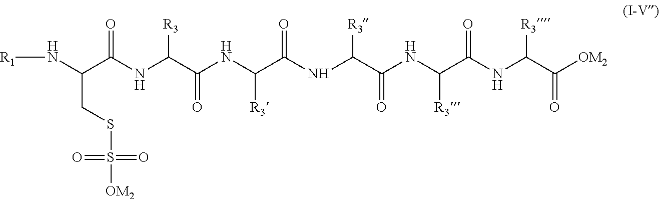

where
R1 stands for a hydrogen atom or for a structural element of formula (II)

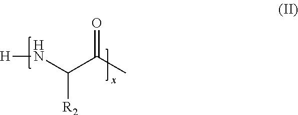

where
x stands for an integer from 1 to about 10,
the R2 radical in each of the structural elements of formula (II) may be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3, R3′, R3″, R3″ and R3″″ stands for a 2-carboxyethyl group (i.e., an HOOC—CH₂—CH₂— group) and
the other radicals of R3, R3′, R3″, R3″ and R3″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another most especially preferred embodiment, a multicomponent packaging unit as contemplated herein for permanent shaping of keratinic fibers is characterized in that the agent (B) in container (ii) contains at least one compound of formula (I-VI″) wherein

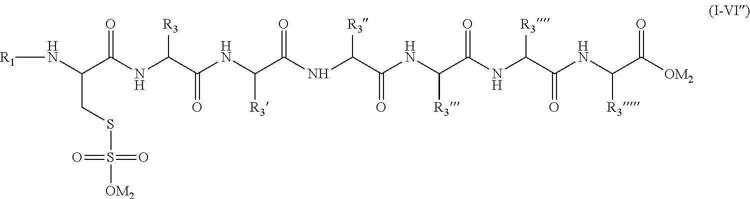

where
R1 stands for a hydrogen atom or for a structural element of formula (II)

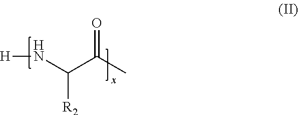

where
x stands for an integer from 1 to about 10,
the R2 radical in each of the structural elements of formula (II) may be selected independently of the preceding structural element of formula (II), R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
one of the radicals of R3, R3′, R3″, R3′″, R3″″ and R3″″′ stands for a 2-carboxyethyl group (i.e., an HOOC—CH₂—CH₂— group) and
the other radicals of R3, R3′, R3″, R3′″, R3″″ and R3″″, independently of one another, stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M2 stands for a hydrogen atom, one equivalent of a monovalent or polyvalent cation or an ammonium ion (NH₄)⁺.

In another most especially preferred embodiment, a multicomponent packaging unit as contemplated herein is characterized in that the agent (B) in container (ii) contains at least one compound of formula (I) wherein

M1 stands for a structural element of formula (III), and
y stands for an integer of at least 3, and
the R3 radical in at least three structural element of formula (III) stands for a 2-carboxyethyl group (i.e., an HOOC—CH₂—CH₂— group).

In another most especially preferred embodiment, a multicomponent packaging unit as contemplated herein for permanent shaping of keratinic fibers is characterized in that the agent (B) in container (ii) contains at least one compound of formula (I) wherein

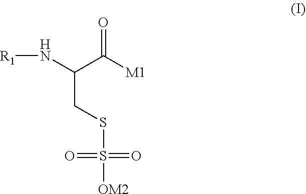

where
R1 stands for a structural element of formula (II)

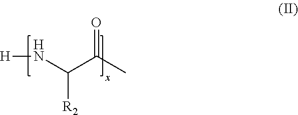

where
x stands for an integer from 1 to about 10,
the R2 radical in each of the structural elements of formula (II) may be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group and
M1 stands for a structural element of formula (III):

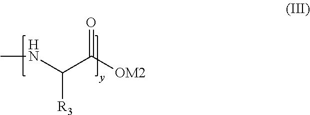

where
y stands for an integer from 3 to about 10,
the R3 radical in each of the structural elements of formula (III) may be selected independently of the preceding structural element of formula (III),
the R3 radical in at least three structural elements of formula (III) stands for a 2-carboxyethyl group (i.e., a group [sic] (i.e., an HOOC—CH₂—CH₂— group) and
the R3 radical in the remaining structural elements of formula (III) stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoyl ethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

In another most especially preferred embodiment, a multicomponent packaging unit as contemplated herein for permanent shaping of keratinic fibers is characterized in that the agent (B) in container (ii) contains at least one compound of formula (I) wherein

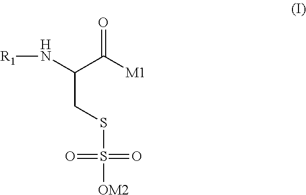

where
R1 stands for a structural element of formula (II)

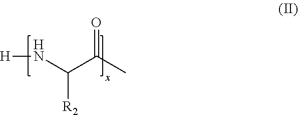

where
x stands for an integer from about 8 to about 10,
the R2 radical in each of the structural elements of formula (II) may be selected independently of the preceding structural element of formula (II),
one of the R2 radicals stands for a 2-methylpropyl group and
one of the R2 radicals stands for an isopropyl group and
one of the R2 radicals stands for a 3-carbamimidamidopropyl group (i.e., a H₂N—C(NH)—NH—CH₂—CH₂—CH₂— group) and
one of the R2 radicals stands for a 1-methylpropyl group (i.e., a H₃C—CH₂—CH(CH₃)— group) and
three of the R2 radicals stand for a 2-carboxyethyl group (i.e., an HOOC—CH₂—CH₂— group) and
one of the R2 radicals stands for a 4-hydroxybenzyl group (i.e., a 4-OH—C₆H₅—CH₂— group)
the additional R2 radicals stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
   M1 stands for the —OM2 group or for a structural element of formula (III):

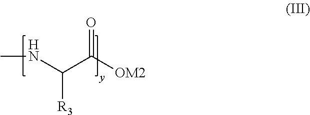

where
y stands for an integer from 1 to about 10,
the R3 radical in each of the structural elements of formula (III) may be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺ and
(b) at least one keratin-reducing compound which has at least one thiol group (—SH) and/or at least one disulfide (—S—S—).

In another most especially preferred embodiment, a multicomponent packaging unit as contemplated herein for permanent shaping of keratinic fibers is characterized in that the agent (B) in container (ii) contains at least one compound of formula (I) wherein

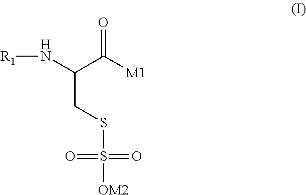

where
R1 stands for a hydrogen atom or for a structural element of formula (II)

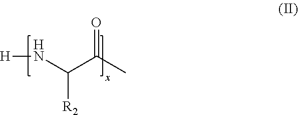

where
x stands for an integer from 1 to about 10,
the R2 radical in each of the structural elements of formula (II) may be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for a structural element of formula (III):

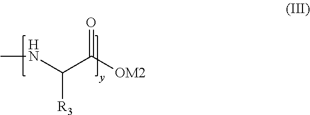

where
y stands for an integer from about 8 to about 10,
the R3 radical in each of the structural elements of formula (II) may be selected independently of the preceding structural element of formula (II),
one of the R3 radicals stands for a 2-methylpropyl group and
one of the R3 radicals stands for an isopropyl group and
one of the R3 radicals stands for a 3-carbamimidamidopropyl group (i.e., a H₂N—C(NH)—NH—CH₂—CH₂—CH₂— group) and
one of the R3 radicals stands for a 1-methylpropyl group (i.e., a H₃C—CH₂—CH(CH₃)— group) and
two of the R3 radicals stand for a 2-carboxyethyl group (i.e., an HOOC—CH₂—CH₂— group) and
one of the R3 radicals stands for a 4-hydroxybenzyl group (i.e., a 4-OH—C₆H₅—CH₂— group)
the additional R3 radicals stand for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
   M2 stands for a hydrogen atom, one equivalent of a monovalent or polyvalent cation or an ammonium ion (NH₄)⁺.

The compounds of formula (I) may be used in a total amount of from about 0.0001 to about 10% by weight—based on the weight of the agent (B). However, it has surprisingly been found that the compound(s) of formula (I) can achieve a very good reduction in hair damage and an improvement in the waving effect even in low use concentrations. For this reason it is particularly advantageous if the agent (B) contains one or more compounds of formula (I) in a total amount of from about 0.001 to about 10.0% by weight, preferably from about 0.01 to about 2.5% by weight, more preferably from about 0.05 to about 1.0% by weight and especially preferably from about 0.05 to about 0.3% by weight—based on its total weight.

In an additional most especially preferred embodiment, a multicomponent packaging unit as contemplated herein is characterized in that the agent (B) in container (ii) contains one or more compounds of formula (I) in a total amount of from about 0.0001 to about 10% by weight, preferably from about 0.001 to about 10.0% by weight, more preferably from about 0.01 to about 2.5% by weight, even more preferably from about 0.05 to about 1.0% by weight and especially preferably from about 0.05 to about 0.3% by weight—based on the total weight of the agent (B).

### Multicomponent Packaging Unit (Kit of Parts) Additional Components(s)

The agent (A) described above is an agent for permanent shaping of hair, the agent being applied to the hair to reduce the cystine-disulfide bonds in the hair. The hair is usually shaped mechanically before, during or after application of the reducing agent formulation.

Agent (B) contains the Bunte salt(s) of formula (I) described above. Agent (B) may be applied before, during or after application of agent (A), for example. In this context, various application processes are possible and preferred.

The hair is shaped mechanically. Agent (A) is then applied to the hair and rinsed out again subsequently. Agent (B) is applied after that.

The hair is shaped mechanically. Agent (A) is then applied to the hair. Agent (B) is applied after that, but without having first rinsed agent (A) out of the hair.

If hair has been wound onto curlers to shape it mechanically, an enhanced waving effect can be achieved by employing the application methods described above. Furthermore, the curls are stable for a longer period of time, i.e., the curls would not straighten out so quickly.

To secure the shaped strands of hair in their form, a fixative solution is next applied to the hair after washing out the reducing agent (and/or agent (B)).

The fixative solution contains an oxidizing agent (usually hydrogen peroxide) and ensures reoxidation of the cysteine components of hair that were still present in thiol form up to this point in time.

In principal the oxidizing agent may also be added to the agent (B) so that the agent (B) at the same time contains the Bunte salt(s) of formula (I) and the oxidizing agent (in particular hydrogen peroxide). However, it has preferably been discovered that the fixative solution in the form of an additional third preparation packaged separately may be added to the multicomponent packaging unit. Within the context of this additional most especially preferred embodiment, the multicomponent packaging unit as contemplated herein therefore also comprises a third container (iii), which contains the fixative agent (C), in addition to containers (i) and (ii), which contain agents (A) and (B).

A multicomponent packaging unit as contemplated herein comprising the following packaged separated is most especially preferred:
a container (iii) containing a cosmetic agent (C), wherein
the agent (C) contains at least one oxidizing agent.

The fixative agent (i.e., the cosmetic agent (C)) contains the oxidizing agent, in particular hydrogen peroxide, in a cosmetic vehicle. In a preferred embodiment, hydrogen itself is used as an aqueous solution in the fixative agent (C). The concentration of hydrogen peroxide solution is determined here on the one hand by the statutory provisions and on the other hand by the desired effect; from about 1.5 to about 12% by weight solutions in water are preferred for use. Fixative agents (C) preferred as contemplated herein are characterized in that they contained from about 1.0% to about 15.0% by weight, preferably from about 2.0% to about 12.0% by weight, more preferably from about 3.0% to about 9.0% by weight and most especially preferably from about 3.5% to about 7.5% by weight hydrogen peroxide, each based on the total weight of the fixative agent (C).

In another particularly preferred embodiment, a multicomponent packaging unit as contemplated herein is characterized in that the agent (C) in container (iii) contains hydrogen peroxide as the oxidizing agent.

In another particularly preferred embodiment, a multicomponent packaging unit as contemplated herein is characterized in that the agent (C) in container (iii) contains from about 1.0 to about 15.0% by weight, preferably from about 2.0 to about 12.0% by weight, more preferably from about 3.0 to about 9.0% by weight and most especially preferably from about 3.5 to about 7.5% by weight hydrogen peroxide—based on the total weight of the agent (C). In addition to containers (i) and (ii) (and optionally container (iii)) the multicomponent packaging unit as contemplated herein may also comprise at least one additional container. This fourth container (iv) may contain, for example, agent (D) which may be an additional pretreatment agent and/or after-treatment agent. Agent (D) may be a shampoo, a conditioner or also an agent of a care droplet type, wherein the care droplets can be mixed with the agent (A) and/or (B) (and/or (C)) prior to use, for example.

This fourth agent (D) is most especially preferably an aqueous care formulation adjusted to be acidic.

In another embodiment a multicomponent packaging unit comprising the following packaged separately is therefore particularly preferred:
a container (iv) containing a cosmetic agent (D),
   wherein
the agent (D) contains at least one acid in an aqueous cosmetic vehicle and has a pH of from about 1.5 to about 5.5, preferably of from about 2.0 to about 5.0, more preferably of from about 2.5 to about 4.5 and most especially preferably from about 2.5 to about 4.0.

It has proven preferable for agent (A) to be adjusted to an alkaline pH which is advantageously in the range of from about 7.0 to about 10.0. To adjust the alkaline pH, the alkalizing agents known from cosmetics and approved for cosmetic may be used. For example, ammonia, C₁-C₄ alkanolamines or inorganic hydroxides are particularly suitable.

The agents (B), (C) and/or (D) are preferably adjusted to a neutral to acidic pH. The acidic pH can be adjusted in principle by using various acids. Acids that have proven to be suitable include, for example, citric acid, tartaric acid, malic acid, lactic acid, acetic acid, sulfuric acid, hydrochloric acid, phosphoric acid, methane sulfonic acid, benzoic acid, malonic acid, oxalic acid and/or 1-hydroxyethane-1,1-diphosphonic acid. Preferred acids are those from the group of citric acid, tartaric acid, malic acid, lactic acid, methane sulfonic acid, oxalic acid, malonic acid, benzoic acid, hydrochloric acid, sulfuric acid, phosphoric acid and/or 1-hydroxyethane-1,1-diphosphonic acid.

In another most especially preferred embodiment, a multicomponent packaging unit as contemplated herein is characterized in that
agent (A) in container (i) contains water and has a pH of from about 7.0 to about 10.0, preferably from about 7.5 to about 9.5,
agent (B) in container (ii) contains water and has a pH of from about 2.5 to about 6.5, preferably from about 3.0 to about 5.5, and
agent (C) in container (iii) contains water and has a pH of from about 1.5 to about 5.5, preferably from about 2.5 to about 4.5.

All the agents described above—i.e., agents (A) and (B) as well as the other agents (C) and (D) that are optionally present—may also contain other ingredients. Thus, for example, the agents may additionally contain organic solvents such as methoxybutanol, ethyl diglycol, 1,2-propylene glycol, n-propanol, n-butanol, n-butylene glycol, glycerol, diethylene glycol monoethyl ether and diethylene glycol mono-n-butyl ether. Preferred water-soluble organic solvents include all those in which the solvent is present in a total amount of from about 0.1% to about 30% by weight, preferably from about 1% to about 20% by weight, in particular from about 2% to about 10% by weight, based on the total weight of the respective cosmetic agent.

The agents—i.e., agents (A) and (B) as well as the other agents (C) and (D) that are optionally present—may additional contain at least one other conditioning substance selected from the group of cationic polymers, cationic surfactants, silicones, solids as well as mixtures thereof. The term “conditioning substance” is understood as contemplated herein to refer to substances which are absorbed by keratinic materials in particular hair and improve the physical and sensory properties of both hair and also of the product as such. Conditioning agents smooth the top layer of hair and make it soft and supple.

Preferred cationic polymers are selected from the group of poly(methacryloyloxyethyl trimethylammonium chloride) (INCI Polyquaternium-37), quaternized cellulose derivatives (INCI Polyquaternium 10), cationicalkyl polyglycosides, cationized honey, cationic guar derivatives, polymeric dimethyldiallylammonium salts and copolymers thereof with esters and amides of acrylic acid and methacrylic acid, copolymers of vinylpyrrolidone with quaternized derivatives of dialkylaminoalkyl acrylate and methacrylate, vinylpyrrolidonevinylimidazolium methochloride copolymers, quaternized polyvinyl alcohol, Polyquaternium 2, Polyquaternium 7, Polyquaternium 17, Polyquaternium 18, Polyquaternium 24, Polyquaternium 27 as well as mixtures thereof.

Within the scope of the present disclosure it may be preferable if the cationic polymer(s) is/are present in a total amount of from about 0.05% to about 7.5% by weight, preferably from about 0.1% to about 5% by weight, especially from about 0.2% to about 3.5% by weight, in particular from about 0.25% to about 2.5% by weight, each based on the total weight of the respective cosmetic agent.

Suitable conditioning substances within the scope of the present disclosure also include cationic surfactants from the group of quaternary ammonium compounds and/or ester quats and/or amidoamines.

Preferred cationic surfactants are selected from the group of alkyltrimethylammonium chlorides, preferably with 10 to 18 carbon atoms in the alkyl radical, dialkyldimethylammonium chlorides preferably with 10 to 18 carbon atoms in the alkyl radical, trialkylmethylammonium chlorides preferably with 10 to 18 carbon atoms in the alkyl radical, cetyltrimethylammonium chloride, stearyltrimethylammonium chloride, di stearyldimethylammonium chloride, lauryldimethylammonium chloride, lauryl dimethyl benzyl ammonium chloride, tricetylmethylammonium chloride, Quaternium-27, Quaternium-83, N-methyl-N(2-hydroxy-ethyl)-N,N-(di-tallow acyloxyethyl)ammonium methosulfate, N-methyl-N(2-hydroxyethyl)-N,N-(distearoyloxyethyl)ammoniummethosulfate, N,N-dimethyl-N,N-distearoyloxyethylammonium chloride, N,N-di-(2-hydroxyethyl)-N,N-(fatty acid ester ethyl)ammonium chloride as well as mixtures thereof.

The cation surfactant(s) is/are preferably present in a total amount of from about 0.5% to about 50% by weight in the cosmetic agents used as contemplated herein, especially from about 1% to about 40% by weight, especially preferably from about 1.5% to about 30% by weight, in particular from about 2% to about 20% by weight, each based on the total weight of the respective cosmetic agent.

Agents suitable as contemplated herein are characterized in that the agent additionally contains at least one anionic surfactant. Preferred anionic surfactants include fatty acids, alkyl sulfates, alkyl ether sulfates and ether carboxylic acids with from about 10 to about 20 carbon atoms in the alkyl group and up to about 16 glycol ether groups in the molecule.

Suitable agents as contemplated herein are characterized in that the agent additionally contains at least zwitterionic surfactant. Preferred zwitterionic surfactants include betaine, N-alkyl-N,N-dimethylammonium glycinates, N-acyl-aminopropyl-N,N-dimethylammonium glycinate and 2-alkyl-3-carboxymethyl-3-hydroxyethylimidazolines. A preferred zwitterionic surfactant is known by the INCI designation cocamidopropyl betaine.

Agents suitable as contemplated herein are characterized in that the agent additionally contains at least one amphoteric surfactant. Preferred amphoteric surfactants include N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids. Especially preferred amphoteric surfactants include N-cocoalkylaminopropionate, cocoacylaminoethylaminopropionate and C₁₂-C₁₈ acylsarcosine.

In addition it has proven advantageous if the agents contain additional nonionic surfactant substances. Preferred nonionic surfactants include alkyl polyglycosides and alkylene oxide addition products onto fatty alcohols and fatty acids each with from about 2 to about 30 mol ethylene oxide per mol fatty alcohol and/or fatty acid. Preparations with excellent properties are also obtained if they contain fatty acid esters of ethoxylated glycerol as the nonionic surfactants.

The nonionic, zwitterionic or amphoteric surfactants are used in amounts of from about 0.1 to about 45% by weight, preferably from about 1 to about 30% by weight and most especially preferably from about 1 to about 15% by weight, based on the total weight of the respective agent.

The agents as contemplated herein may also contain at least one thickener. There are no fundamental restrictions with respect to these thickeners. Both organic and purely inorganic thickeners may also be used.

Suitable thickeners include anionic synthetic polymers, cationic synthetic polymers, naturally occurring thickeners such as nonionic guar gum, scleroglucan gum or xanthan gum, gum arabic, ghatti gum, karaya gum, tragacanth gum, carrageenan gum, agar, pectins, alginates, starch fractions and derivatives such as amylose, amylopectin and dextrins as well as cellulose derivatives such as methylcellulose, carboxyalkyl celluloses and hydroxyalkyl celluloses, for example, nonionic fully synthetic polymers such as polyvinyl alcohol or polyvinylpyrrolidone as well as inorganic thickeners, in particular layered silicates such as bentonite, especially smectites such as montmorillonite or hectorite for example.

In addition, it has proven advantageous if the agents as contemplated herein contain at least one stabilizer or chelating agent in particular if they additionally contain hydrogen peroxide. Especially preferred stabilizers include phenacetin, alkali benzoates (sodium benzoate) and salicylic acid. Furthermore, all state-of-the-art chelating agents can be used. Preferred chelating agents as contemplated herein include nitrogen-containing polycarboxylic acids, in particular EDTA and EDDS and phosphonates in particular 1-hydroxyethane-1,1-diphosphonate (HEDP) and/or ethylenediamine tetramethylene phosphonate (EDTMP) and/or diethylenetriamine pentamethylene phosphonate (DTPMP) as well as the sodium salts thereof.

Furthermore, the respective agents may also contain active ingredients, excipients and additives, such as nonionic polymers, for example, vinylpyrrolidone/vinyl acrylate copolymers, polyvinylpyrrolidone, vinylpyrrolidone/vinyl acetate copolymers, polyethylene glycols and polysiloxane; additional silicones such as volatile or nonvolatile, linear branched or cyclic crosslinked or non-crosslinked polyalkylsiloxanes (such as dimethicone or cyclomethicone), polyarylsiloxanes and/or polyalkylarylsiloxanes, in particular polysiloxanes with organofunctional groups such as substituted or unsubstituted amines (amodimethicone), carboxyl groups, alkoxy groups and/or hydroxyl groups (dimethicone copolyols), linear polysiloxane (A)-polyoxyalkylene (B) block copolymers, grafted silicone polymers; cationic polymers such as quaternized cellulose ethers, polysiloxanes with quaternary groups, dimethyl diallyl ammonium chloride polymers, acrylamide-dimethyl diallyl ammonium chloride copolymers, dimethylaminoethyl methacrylate-vinyl polypyrrolidone copolymers quaternized with diethyl sulfate, vinylpyrrolidone-imidazolinium-methochloride copolymers and quaternized polyvinyl alcohol; zwitterionic and amphoteric polymers; anionic polymers such as polyacrylic acids, for example; structurants such as glucose, maleic acid and lactic acid, hair conditioning compounds such as phospholipids, for example, lectin and kephalins; perfume oils, dimethyl isosorbide and cyclodextrins; fiber structure improving active ingredients in particular mono-, di- and oligosaccharides such as glucose, galactose, fructose, fruit sugar and lactose; coloring agents for coloring the agent; antidandruff active ingredients, such as piroctones, olamines, zinc omadines and climbazole; amino acids and oligopeptides; protein hydrolysates, based on animal and/or plant materials as well as those in the form of their fatty acid condensation products or optionally anionically or cationally modified derivatives; vegetable oils; light protectants and UV blockers; active ingredients such as panthenol, pantothenic acid, pantolactone, allantoin, pyrrolidone carboxylic acids and the salts thereof as well as bisabolol; polyphenols, in particular hydroxycinnamic acids, 6,7-dihydroxycoumarins, hydroxybenzoic acids, catechols, tannins, leukoanthocyanidines, anthocyanidines, flavanones, flavones and flavonols; ceramides or pseudoceramides; vitamins, provitamins and vitamin precursors; plant extracts; fats and waxes such as fatty alcohols, beeswax, montan wax and paraffins; swelling agents and penetration substances such as glycerol, propylene glycol monoethyl ether, carbonates, bicarbonates, guanidines, ureas as well as primary, secondary and tertiary phosphates; opacifiers such as latex, styrene/PVP and styrene/acrylamide copolymers; pearlizing agents such as ethylene glycol monostearate and distearate as well as PEG-3 distearate; pigments and blowing agents such as propane-butane mixtures, N₂O, dimethyl ether, CO₂ and air.

Those skilled in the art will chose these additional substances in accordance with the desired properties of the agents. Reference is made explicitly to the relevant handbooks with which those skilled in the art are familiar with regard to additional optional components as well as the amounts of these components to be used. The additional active ingredients and excipients are preferably used in the agents as contemplated herein in amounts of from about 0.0001 to about 25% by weight, in particular of from about 0.0005 to about 15% by weight, each based on the total weight of the respective agent.

### Methods of Permanently Changing the Shape of Keratinic Fibers

The agents made available with the multicomponent packaging unit as contemplated herein of the first subject matter of the present disclosure are used in methods for permanently changing the shape of keratinic fibers, in particular human hair.

A third subject matter of the present disclosure is therefore a method for permanently changing the shape of keratinic fibers, in particular human hair, comprising the following steps:

a1) Applying a cosmetic agent (A) to the keratinic fibers,
a2) Allowing the agent (A) to act for a period of from about 2 to about 60 minutes,
a3) Optionally rinsing the agent (A) out of the keratinic fibers,
b1) Applying a cosmetic agent (B) to the keratinic fibers,
b2) Allowing the agent (B) to act for a period of from about 2 to about 60 minutes,
b3) Optionally rinsing the cosmetic agent (B) out of the keratinic fibers,
c1) Applying a cosmetic agent (C) to the keratinic fibers,
c2) Allowing the agent (C) to act for a period of from about 2 to about 60 minutes,
c3) Rinsing the agent (C) out of the keratinic fibers, wherein
the agents (A), (B) and (C) are the agents as disclosed in detail in the description of the first subject matter of the present disclosure.

The agents (A), (B) and (C) are the agents as disclosed in detail in the description of the first subject matter of the present disclosure, i.e., agent (A) is applied from a first container (i) and contains at least one keratin-reducing compound. Agent (B) is applied from a second container (ii) and contains at least one compound of general formula (I). Finally, agent (C) is applied from a third container (iii); it is the fixative and contains at least one oxidizing agent, in particular hydrogen peroxide.

Within this method, various sequences of steps are particularly suitable.

A method comprising the following steps in the order indicated is most especially preferred:

a1) Applying a cosmetic agent (A) to the keratinic fibers,
a2) Allowing the agent (A) to act for a period of from about 2 to about 60 minutes,
a3) Rinsing the agent (A) out of the keratinic fibers,
b1) Applying a cosmetic agent (B) to the keratinic fibers,
b2) Allowing the agent (B) to act for a period of from about 2 to about 60 minutes,
b3) Optionally rinsing the cosmetic agent (B) out of the keratinic fibers,
c1) Applying a cosmetic agent (C) to the keratinic fibers,
c2) Allowing the agent CB) to act for a period of from about 2 to about 60 minutes,
c3) Rinsing the agent (C) out of the keratinic fibers.

Within the scope of this embodiment, for example, the hair is first wound onto curlers, then agent (A) is applied, allowed to act and rinsed out. Within the scope of this embodiment, agent (B) functions as an intermediate treatment agent and is applied to the hair after the reducing agent but before the fixative (C). Agent (B) can be rinsed out but it is also possible not to rinse out the agent (B) but instead to apply the fixative (C) to the hair which is still being treated with agent (B). In this case agents (B) and (C) are then rinsed out together in step c3). When carrying out this method, a further increase in the waving effect can be guaranteed. In addition, it has been found that damage to the hair can be reduced in this way.

A method comprising the following steps in the order indicated is also preferred:

a1) Applying a cosmetic agent (A) to the keratinic fibers,
a2) Allowing the agent (A) to act for a period of from about 2 to about 60 minutes,
b1) Applying a cosmetic agent (B) to the keratinic fibers,
b2) Allowing the agent (B) to act for a period of from about 2 to about 60 minutes,
b3) Rinsing the cosmetic agents (A) and (B) out of the keratinic fibers,
c1) Applying a cosmetic agent (C) to the keratinic fibers,
c2) Allowing the agent (C) to act for a period of from about 2 to about 60 minutes,
c3) Rinsing the agent (C) out of the keratinic fibers.

Within the scope of this embodiment, for example, the hair is first wound onto curlers, then agent (A) is applied and allowed to act. However, agent (A) is not rinsed out but instead agent (B) is then applied to the hair—which is still being treated with agent (A). Then in step b3), agent (A) and agent (B) are rinsed out of the hair together. After rinsing out agents (A)/(B), the fixative (C) is then again applied to the hair, allowed to act and rinsed out.

In addition a method comprising the following steps in the order indicated is also particularly preferred:

a1) Applying a cosmetic agent (A) to the keratinic fibers,
a2) Allowing the agent (A) to act for a period of from about 2 to about 60 minutes,
a3) Rinsing the agent (A) out of the keratinic fibers,
c1) Applying a cosmetic agent (C) to the keratinic fibers,
c2) Allowing the agent (C) to act for a period of from about 2 to about 60 minutes,
c3) Rinsing the agent (C) out of the keratinic fibers,
b1) Applying a cosmetic agent (B) to the keratinic fibers,
b2) Allowing the agent (B) to act for a period of from about 2 to about 60 minutes,
b3) Optionally rinsing the cosmetic agent (B) out of the keratinic fibers.

Within the scope of this embodiment the keratin fibers, preferably hair, are wound onto curlers and shaped in the conventional way by successive application of agent (A) (permanent wave agent) and (C) (fixative). Next the agent (B) is applied to the fibers (hair) as an after-treatment agent. In this context, agent (B) can be packaged as a “leave-on” agent as well as being a “rinse-off” agent. If agent (B) is packaged as a leave-on agent, it remains on the keratin fibers (hair) after application. If agent (B) is packaged as a rinse-off agent, it is rinsed out after being applied and allowed to act.

A method comprising the following steps in the order indicated is also suitable and inventive:

a1) Applying a cosmetic agent (A) to the keratinic fibers,
a2) Allowing the agent (A) to act for a period of from about 2 to about 60 minutes,
a3) Rinsing the agent (A) out of the keratinic fibers,
c1) Applying a cosmetic agent (C) to the keratinic fibers,
c2) Allowing the agent (C) to act for a period of from about 2 to about 60 minutes,
b1) Applying a cosmetic agent (B) to the keratinic fibers,
b2) Allowing the agent (B) to act for a period of from about 2 to about 60 minutes,
b3) Rinsing the cosmetic agents (B) and (C) out of the keratinic fibers.

Within the scope of this embodiment, the keratin fibers, preferably hair, are wound onto curlers and shaped in the conventional way by successive application of agents (A) (permanent wave agent) and (C) (fixative). However, agent (C) is not rinsed out before applying agent (B). In this case, agents (A) and (B) are both rinsed out together in step b3).

The multicomponent packaging units and methods for permanent shaping described previously are capable of straightening keratin fibers as well as giving them a permanent wave.

For a permanent wave, the hair may preferably be rolled onto foil or curlers before applying agent (A). For straightening hair, the hair is preferably combed or brushed straight with comb or a brush while the agent (A) is active or after it has been rinsed out.

In a particularly preferred embodiment, the method as contemplated herein is therefore characterized in that the keratinic fibers are shaped with the aid of shaping agents before, during or after step a1), a2) or a3).

Shaping aids include for example, curlers, foil, combs, brushes.

With respect to the embodiments of the method as contemplated herein that are particularly preferred, what was said about the multicomponent packaging units as contemplated herein also applies, mutatis mutandis.

### Use

Another subject matter of the present disclosure is finally the use of a compound of formula (I)

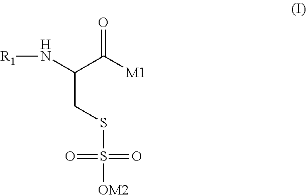

where the R1, M1 and M2 radicals have the meanings as defined in claim 1, to reduce the hair damage caused by keratin-reducing compounds.

Hair damage can be ascertained and/or quantified by employing various physical measurement methods. For example, the cysteine value of the hair can be determined by IR measurements. The stress-strain behavior of hair can be measured or the hair can be evaluated with respect to its combability or the extent of the resulting split ends and hair breakage.

With respect to the particularly preferred embodiments of use of the compounds of formula (I), what was said regarding the multicomponent packaging units as contemplated herein also applies here, mutatis mutandis.

### EXAMPLES

### 1. Formulations

The following compositions were prepared (all quantitative amounts in wt %)

Agent (A)

| | A1 | A2 | A3 |
|---|---|---|---|
| Ammonium thioglycolate | 11.8 | 9.5 | 10.2 |
| Turpinal SL | 0.30 | 0.30 | 0.30 |
| Ammonium carbonate | 2.50 | 2.50 | 2.50 |
| Cremophor CO 40 | 0.75 | 0.75 | 0.75 |
| Protelan VE/K | 0.80 | 0.80 | 0.80 |
| Ammonia (25% aqueous solution) | to pH | to pH | to pH |
| | 8.3 | 8.3 | 8.3 |
| Perfume | 0.50 | 0.50 | 0.50 |
| Water | to | to | to |
| | 100 | 100 | 100 |

Cremophor ® CO40 Hydrogenated castor oil with approx. 40-45 EO units (INCI designation: PEG-40 hydrogenated castor oil) (BASF)
Protelan ® VE/K N-Cocoyl wheat protein condensate (INCI designation: sodium cocoyl hydrolyzed wheat protein (Zschimmer & Schwarz)
Texapon ® NSO UP Lauryl ether sulfate, sodium salt (approx. 27.5% active substance; INCI designation: sodium laureth sulfate) (Cognis)

Agent (B)

| | B1 | B2 | B3 |
|---|---|---|---|
| Citric acid (anhydrous) | 0.5 | 0.5 | 0.5 |
| Sodium lauryl ether sulfate (25% aqueous solution) | 50.0 | 50.0 | 50.0 |
| Disodium dicocoampo diacetate | 7.0 | 7.0 | 7.0 |
| Salicylic acid | 0.2 | 0.2 | 0.2 |
| Sodium benzoate | 0.5 | 0.5 | 0.5 |
| Euperlan PK 300 AM | 2.0 | 2.0 | 2.0 |
| (approx. 60-64% solids; INCI: glycol distearate, | | | |
| glycerin, laureth-4, cocamidopropyl betaine (Cognis)) | | | |
| Cetiol HE | 1.0 | 1.0 | 1.0 |
| (cocomonoglyceride with approx. 7.3 EO units (INCI: | | | |
| PEG-7 glyceryl cocoate) (Cognis)) | | | |
| Oligopeptide^{#} | 0.01 | 0.1 | 1.0 |
| Polyquaternium-10 | 0.5 | 0.5 | 0.5 |
| PEG-40 Hydrogenated castor oil | 1.0 | 1.0 | 1.0 |
| Sodium chloride | 0.5 | 0.5 | 0.5 |
| Water | to | to | to |
| | 100 | 100 | 100 |

^{#}Oligopeptide mixture of formula (I), where R1 = formula (II), M1 = formula (III),
x = 1-10 and y = 1-10, M2 = hydrogen, average molecular weight 400-800 Dalton

| | B4 | B5 | B6 |
|---|---|---|---|
| Citric acid (anhydrous) | 0.5 | 0.5 | 0.5 |
| Sodium lauryl ether sulfate (25% aqueous solution) | 50.0 | 50.0 | 50.0 |
| Disodium dicocoampo diacetate | 7.0 | 7.0 | 7.0 |
| Salicylic acid | 0.2 | 0.2 | 0.2 |
| Sodium benzoate | 0.5 | 0.5 | 0.5 |
| Euperlan PK 300 AM | 2.0 | 2.0 | 2.0 |
| (approx. 60-64% solids; INCI: glycol distearate, | | | |
| glycerin, laureth-4, cocamidopropyl betaine (Cognis)) | | | |
| Cetiol RE | 1.0 | 1.0 | 1.0 |
| (cocomonoglyceride with approx. 7.3 EO units (INCI: | | | |
| PEG-7 glyceryl cocoate) (Cognis)) | | | |
| 2-Amino-3-(sulfosulfanyl)propanoic acid | 0.01 | 0.1 | 1.0 |
| Polyquaternium-10 | 0.5 | 0.5 | 0.5 |
| PEG-40 Hydrogenated castor oil | 1.0 | 1.0 | 1.0 |
| Sodium chloride | 0.5 | 0.5 | 0.5 |
| Water | to | to | to |
| | 100 | 100 | 100 |
| | B7 | B7 | B9 |
| Liquid paraffin | 1.0 | 1.0 | 1.0 |
| Dehyquart F75 (INCI designation: distearoylethyl | 2.0 | 2.0 | 2.0 |
| hydroxyethylmonium methosulfate, cetearyl alcohol) | | | |
| Oligopeptide^{#} | 0.01 | 0.1 | 1.0 |
| Quaternium-87 | 1.5 | 1.5 | 1.5 |
| Cetearyl alcohol | 3.5 | 3.5 | 3.5 |
| Propylparaben | 0.15 | 0.15 | 0.15 |
| Cetylpalmitate | 0.7 | 0.7 | 0.7 |
| Stearamidopropyldimethylamine | 1.0 | 1.0 | 1.0 |
| Trimethylhexadecylammonium chloride | 0.6 | 0.6 | 0.6 |
| Citric acid | 0.5 | 0.5 | 0.5 |
| Methylparaben | 0.15 | 0.15 | 0.15 |
| Water | to | to | to |
| | 100 | 100 | 100 |

^{#}Oligopeptide mixture of formula (I), where R1 = formula (II), M1 = formula (III),
x = 1-10 and y = 1-10, average molecular weight 400-800 Dalton

| | B10 | B11 | B12 |
|---|---|---|---|
| Liquid paraffin | 1.0 | 1.0 | 1.0 |
| Dehyquart F75 (INCI designation: distearoylethyl | 2.0 | 2.0 | 2.0 |
| hydroxyethylmonium methosulfate, cetearyl | | | |
| alcohol) | | | |
| 2-Amino-3-(sulfosulfanyl)propanoic acid | 0.01 | 0.1 | 1.0 |
| Quaternium-87 | 1.5 | 1.5 | 1.5 |
| Cetearyl alcohol | 3.5 | 3.5 | 3.5 |
| Propylparaben | 0.15 | 0.15 | 0.15 |
| Cetyl palmitate | 0.7 | 0.7 | 0.7 |
| Stearamidopropyldimethylamine | 1.0 | 1.0 | 1.0 |
| Trimethylhexadecylammonium chloride | 0.6 | 0.6 | 0.6 |
| Citric acid | 0.5 | 0.5 | 0.5 |
| Methylparaben | 0.15 | 0.15 | 0.15 |
| Water | to | to | to |
| | 100 | 100 | 100 |

Agent (C)

| | C1 | C2 | C3 |
|---|---|---|---|
| Dipicolinic acid | 0.10 | 0.10 | 0.10 |
| Turpinal SL | 1.70 | 1.70 | 1.70 |
| Texapon NSO UP | 6.00 | 6.00 | 6.00 |
| Hydrogen peroxide | 4.00 | 5.00 | 7.5 |
| Ammonia (25% aqueous solution) | 0.70 | 0.70 | 0.70 |
| Water | to | to | to |
| | 100 | 100 | 100 |

Method with sequence of steps in the order indicated

| | 1 | 2 | 3 | 4 | 5 | 6 |
|---|---|---|---|---|---|---|
| | Wind hair | Wind hair | Wind hair | Wind hair | Wind hair | Wind hair |
| | on curlers | on curlers | on curlers | on curlers | on curlers | on curlers |
| a1) | Apply agent | Apply agent | Apply agent | Apply agent | Apply agent | Apply agent |
| | (A1) | (A1) | (A2) | (A2) | (A3) | (A3) |
| a2) | Treat with | Treat 30 | Treat 20 | Treat 20 | Treat with | Treat with |
| | (A1) 30 min | min | min | min | (A3) 40 min | (A3) 40 min |
| a3) | Rinse out | Rinse out | Rinse out | Rinse out | Rinse out | Rinse out |
| | (A1) | (A1) | (A2) | (A2) | (A3) | (A3) |
| b1) | Apply agent | Apply agent | Apply agent | Apply agent | Apply agent | Apply agent |
| | (B1) | (B1) | (B2) | (B2) | (B3) | (B3) |
| b2) | Treat with | Treat with | Treat with | Treat with | Treat with | Treat with |
| | (B1) 30 min | (B1) 30 min | (B2) 10 min | (B2) 10 min | (B3) 20 min | (B3) 20 min |
| b3) | Rinse out | Do not rinse | Rinse out | Do not rinse | Rinse out | Do not rinse |
| | (B1) | out (B1) | (B2) | out (B2) | (B3) | out (B3) |
| c1) | Apply agent | Apply agent | Apply agent | Apply agent | Apply agent | Apply agent |
| | (C1) | (C1) | (C2) | (C2) | (C3) | (C3) |
| c2) | Treat with | Treat with | Treat with | Treat with | Treat with | Treat with |
| | (C1) 30 min | (C1) 30 min | (C2) 20 min | (C2) 20 min | (C3) 15 min | (C3) 15 min |
| c3) | Rinse out | Rinse out | Rinse out | Rinse out | Rinse out | Rinse out |
| | (C1) | (C1) | (C2) | (C2) | (C3) | (C3) |
| | 7 | 8 | 9 | 10 | 11 | 12 |
| | Wind hair | Wind hair | Wind hair | Wind hair | Wind hair | Wind hair |
| | on curlers | on curlers | on curlers | on curlers | on curlers | on curlers |
| a1) | Apply agent | Apply agent | Apply agent | Apply agent | Apply agent | Apply agent |
| | (A1) | (A1) | (A2) | (A2) | (A3) | (A3) |
| a2) | Treat with | Treat 30 | Treat 20 | Treat 20 | Treat with | Treat with |
| | (A1) 30 min | min | min | min | (A3) 40 min | (A3) 40 min |
| a3) | Rinse out | Rinse out | Rinse out | Rinse out | Rinse out | Rinse out |
| | (A1) | (A1) | (A2) | (A2) | (A3) | (A3) |
| b1) | Apply agent | Apply agent | Apply agent | Apply agent | Apply agent | Apply agent |
| | (B4) | (B4) | (B5) | (B5) | (B6) | (B6) |
| b2) | Treat with | Treat with | Treat with | Treat with | Treat with | Treat with |
| | (B4) 30 min | (B4) 30 min | (B5) 10 min | (B5) 10 min | (B6) 20 min | (B6) 20 min |
| b3) | Rinse out | Do not rinse | Rinse out | Do not rinse | Rinse out | Do not rinse |
| | (B4) | out (B4) | (B5) | out (B5) | (B6) | out (B6) |
| c1) | Apply agent | Apply agent | Apply agent | Apply agent | Apply agent | Apply agent |
| | (C1) | (C1) | (C2) | (C2) | (C3) | (C3) |
| c2) | Treat with | Treat with | Treat with | Treat with | Treat with | Treat with |
| | (C1) 30 min | (C1) 30 min | (C2) 20 min | (C2) 20 min | (C3) 15 min | (C3) 15 min |
| c3) | Rinse out | Rinse out | Rinse out | Rinse out | Rinse out | Rinse out |
| | (C1) | (C1) | (C2) | (C2) | (C3) | (C3) |
| | 13 | 14 | 15 | 16 | 17 | 18 |
| | Wind hair | Wind hair | Wind hair | Wind hair | Wind hair | Wind hair |
| | on curlers | on curlers | on curlers | on curlers | on curlers | on curlers |
| a1) | Apply agent | Apply agent | Apply agent | Apply agent | Apply agent | Apply agent |
| | (A1) | (A1) | (A2) | (A2) | (A3) | (A3) |
| a2) | Treat with | Treat 20 | Treat 40 | Treat 40 | Treat with | Treat with |
| | (A1) 20 min | min | min | min | (A3) 30 min | (A3) 30 min |
| a3) | Rinse out | Rinse out | Rinse out | Rinse out | Rinse out | Rinse out |
| | (A1) | (A1) | (A2) | (A2) | (A3) | (A3) |
| c1) | Apply agent | Apply agent | Apply agent | Apply agent | Apply agent | Apply agent |
| | (C1) | (C1) | (C2) | (C2) | (C3) | (C3) |
| c2) | Treat with | Treat with | Treat with | Treat with | Treat with | Treat with |
| | (C1) 40 min | (C1) 40 min | (C2) 30 min | (C2) 20 min | (C3) 20 min | (C3) 20 min |
| c3) | Rinse out | Rinse out | Rinse out | Rinse out | Rinse out | Rinse out |
| | (C1) | (C1) | (C2) | (C2) | (C3) | (C3) |
| b1) | Apply agent | Apply agent | Apply agent | Apply agent | Apply agent | Apply agent |
| | (B7) | (B7) | (B8) | (B8) | (B9) | (B9) |
| b2) | Treat with | Treat with | Treat with | Treat with | Treat with | Treat with |
| | (B7) 30 min | (B7) 30 min | (B8) 10 min | (B8) 10 min | (B9) 20 min | (B9) 20 min |
| b3) | Rinse out | Do not rinse | Rinse out | Do not rinse | Rinse out | Do not rinse |
| | (B7) | out (B7) | (B8) | out (B8) | (B9) | out (B9) |
| | 19 | 20 | 21 | 22 | 23 | 24 |
| | Wind hair | Wind hair | Wind hair | Wind hair | Wind hair | Wind hair |
| | on curlers | on curlers | on curlers | on curlers | on curlers | on curlers |
| a1) | Apply agent | Apply agent | Apply agent | Apply agent | Apply agent | Apply agent |
| | (A1) | (A1) | (A2) | (A2) | (A3) | (A3) |
| a2) | Treat with | Treat 20 | Treat 40 | Treat 40 | Treat with | Treat with |
| | (A1) 20 min | min | min | min | (A3) 30 min | (A3) 30 min |
| a3) | Rinse out | Rinse out | Rinse out | Rinse out | Rinse out | Rinse out |
| | (A1) | (A1) | (A2) | (A2) | (A3) | (A3) |
| c1) | Apply agent | Apply agent | Apply agent | Apply agent | Apply agent | Apply agent |
| | (C1) | (C1) | (C2) | (C2) | (C3) | (C3) |
| c2) | Treat with | Treat with | Treat with | Treat with | Treat with | Treat with |
| | (C1) 40 min | (C1) 40 min | (C2) 30 min | (C2) 20 min | (C3) 20 min | (C3) 20 min |
| c3) | Rinse out | Rinse out | Rinse out | Rinse out | Rinse out | Rinse out |
| | (C1) | (C1) | (C2) | (C2) | (C3) | (C3) |
| b1) | Apply agent | Apply agent | Apply agent | Apply agent | Apply agent | Apply agent |
| | (B710) | (B10) | (B11) | (B11) | (B12) | (B12) |
| b2) | Treat with | Treat with | Treat with | Treat with | Treat with | Treat with |
| | (B10) 30 min | (B10) 30 min | (B11) 10 min | (B11) 10 min | (B12) 20 min | (B12) 20 min |
| b3) | Rinse out | Do not rinse | Rinse out | Do not rinse | Rinse out | Do not rinse |
| | (B10) | out (B10) | (B11) | out (B11) | (B12) | out (B12) |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A multicomponent packaging unit (kit of parts) for permanently changing the shape of keratinic fibers, in particular human hair, comprising the following packaged separately from one another:
a container (i) comprising a cosmetic agent (A) and
a container (ii) comprising a cosmetic agent (B),
wherein
agent (A) in the container (i) comprises at least one keratin-reducing compound and
agent (B) in container (ii) comprises at least one compound of general formula (I) 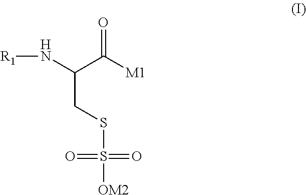
wherein
R1 stands for a hydrogen atom or for a structural element of formula (II) 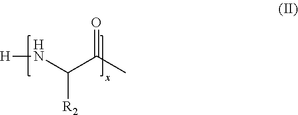
where
x stands for an integer from 1 to about 100,
the R2 radical in each one of the structural elements of formula (II) can each be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III): 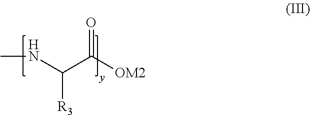
where
y stands for an integer from 1 to about 100,
the R3 radical in each of the structural elements of formula (III) can each be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation or an ammonium ion (NH₄)⁺.

2. The multicomponent packaging unit according to claim 1, wherein the agent (A) in container (i) comprises at least one keratin-reducing compound from the group consisting of thioglycolic acid, thiolactic acid, thiomalic acid, cysteine, cysteamine, cystine, 2-mercaptoethane sulfonic acid and the physiologically tolerable salts thereof.

3. The multicomponent packaging unit according to claim 1, wherein the agent (A) in container (i) comprises one or more keratin-reducing compounds selected from the group consisting of thioglycolic acid, ammonium thioglycolate, thiolactic acid, and ammonium thiolactate.

4. The multicomponent packaging unit according to claim 1, wherein the agent (A) in container (i) comprises—based on the total weight of agent (A)—one or more keratin-reducing compounds in a total amount of from about 1.5 to about 20.0% by weight.

5. The multicomponent packaging unit according to claim 1, wherein the agent (B) in container (ii) comprises at least one compound of formula (I), wherein
R1 stands for a hydrogen atom and
M1 stands for a —OM2 group.

6. The multicomponent packaging unit according to claim 1, wherein the agent (B) in container (ii) comprises at least one compound of formula (I) which has a molecular weight Mw of from about 200 to about 2000 Da.

7. The multicomponent packaging unit according to claim 1, wherein the agent (B) in container (ii) comprises at least one compound of formula (I), wherein
R1 stands for a structural element of formula (II) and
M1 stands for a structural element of formula (III), and
x stands for an integer from 1 to about 10, and
y stands for an integer from 1 to about 10.

8. The multicomponent packaging unit according to claim 1, wherein the agent (B) in container (ii) comprises at least one compound of formula (I) wherein
R1 stands for a structural element of formula (II) and
the R2 radical in at least one structural element of formula (II) stands for a (sulfosulfanyl)methyl group (i.e., an HO—S(O₂)—S—CH₂ group).

9. The multicomponent packaging unit according to claim 1, wherein the agent (B) in container (ii) comprises at least one compound of formula (I) wherein
R1 stands for a structural element of formula (II) and
x stands for an integer of at least 3 and
the R2 radical in at least one structural element of formula (II) stands for a 2-carboxyethyl group.

10. The multicomponent packaging unit according to claim 1, wherein the agent (B) in container (ii) comprises at least one compound of formula (I), wherein
M1 stands for a structural element of formula (III), and
the R3 radical in at least one structural element of formula (III) stands for a (sulfosulfanyl)methyl group (i.e., an HO—S(O₂)—S—CH₂ group).

11. The multicomponent packaging unit according to claim 1, wherein the agent (B) in container (ii) comprises at least one compound of formula (I) wherein
M1 stands for a structural element of formula (III), and
y stands for an integer of at least 3, and
the R3 radical in at least one structural element of formula (III) stands for a 2-carboxyethyl group.

12. The multicomponent packaging unit according to claim 1, wherein the agent (B) in container (ii) comprises—based on the weight of agent (B)—one or more compounds of formula (I) in a total amount of from about 0.0001 to about 10% by weight.

13. The multicomponent packaging unit according to claim 1, comprising, separately packaged—a container (iii) comprising a cosmetic agent (C), wherein agent (C) comprises at least one oxidizing agent.

14. The multicomponent packaging unit according to claim 13, wherein the agent C) in container (iii) comprises hydrogen peroxide as the oxidizing agent.

15. The multicomponent packaging unit according to claim 1, wherein
agent (A) in container (i) comprises water and has a pH of from about 7.0 to about 10.0,
agent (B) in container (ii) comprises water and has a pH of from about 2.5 to about 6.5, and
agent (C) in container (iii) comprises water and has a pH of from about 1.5 to about 5.5.

16. A method for permanently changing the shape of keratinic fibers, comprising the following steps:
a1) Applying a cosmetic agent (A) to the keratinic fibers,
a2) Allowing the agent (A) to act for a period of from about 2 to about 60 minutes,
a3) Optionally rinsing the agent (A) out of the keratinic fibers,
b1) Applying a cosmetic agent (B) to the keratinic fibers,
b2) Allowing the agent (B) to act for a period of from about 2 to about 60 minutes,
b3) Optionally rinsing the agent (B) out of the keratinic fibers,
c1) Applying a cosmetic agent (C) to the keratinic fibers,
c2) Allowing the agent (C) to act for a period of from about 2 to about 60 minutes,
c3) Rinsing the agent (C) out of the keratinic fibers,
wherein
agent (A) is an agent as described in claim 1,
agent (B) is an agent as described in claim 1 and
agent(C) is an agent as described in claim 13.

17. The method according to claim 16, comprising the following steps in the order indicated:
a1) Applying the cosmetic agent (A) to the keratinic fibers,
a2) Allowing the agent (A) to act for a period of from about 2 to about 60 minutes,
a3) Rinsing the agent (A) out of the keratinic fibers,
b1) Applying the cosmetic agent (B) to the keratinic fibers,
b2) Allowing the agent (B) to act for a period of from about 2 to about 60 minutes,
b3) Optionally rinsing the cosmetic agent (B) out of the keratinic fibers,
c1) Applying a cosmetic agent (C) to the keratinic fibers,
c2) Allowing the agent (C) to act for a period of from about 2 to about 60 minutes,
c3) Rinsing the agent (C) out of the keratinic fibers.

18. The method according to claim 16, comprising the following steps in the order indicated:
a1) Applying the cosmetic agent (A) to the keratinic fibers,
a2) Allowing the agent (A) to act for a period of from about 2 to about 60 minutes,
a3) Rinsing the agent (A) out of the keratinic fibers,
c1) Applying the cosmetic agent (C) to the keratinic fibers,
c2) Allowing the agent (C) to act for a period of from about 2 to about 60 minutes,
c3) Rinsing the agent (C) out of the keratinic fibers,
b1) Applying the cosmetic agent (B) to the keratinic fibers,
b2) Allowing the agent (B) to act for a period of from about 2 to about 60 minutes,
b3) Optionally rinsing the cosmetic agent (B) out of the keratinic fibers.

19. The method according to claim 16, wherein
the keratinic fibers are shaped with the assistance of shaping aids before, after or during steps a1), a2) or a3).

20. A method for reducing the damage to hair caused by keratin-reducing compounds, comprising applying a compound of formula (I) onto the hair, 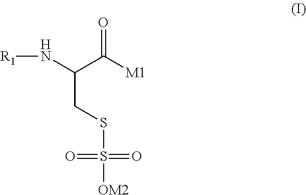
wherein
R1 stands for a hydrogen atom or for a structural element of formula (II) 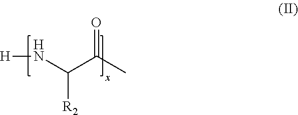
where
x stands for an integer from 1 to about 100,
the R2 radical in each one of the structural elements of formula (II) can each be selected independently of the preceding structural element of formula (II),
R2 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M1 stands for the —OM2 group or for a structural element of formula (III): 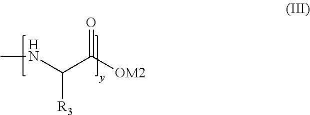
where
v stands for an integer from 1 to about 100,
the R3 radical in each of the structural elements of formula (III) can each be selected independently of the preceding structural element of formula (III),
R3 stands for a hydrogen atom, a methyl group, an isopropyl group, a 2-methylpropyl group, a 1-methylpropyl group, a benzyl group, a 4-hydroxybenzyl group, a hydroxymethyl group, a 1-hydroxyethyl group, a 4-aminobutyl group, a 3-carbamimidamidopropyl group, a 2-carboxyethyl group, a carboxymethyl group, a 2-carbamoylethyl group, a carbamoylmethyl group, a sulfanylmethyl group, a 2-(methylsulfanyl)ethyl group, a 1H-imidazol-4-ylmethyl group, a 1H-indol-3-ylmethyl group or a (sulfosulfanyl)methyl group,
M2 stands for a hydrogen atom, one equivalent of a monovalent or polyvalent cation, or an ammonium ion (NH₄)⁺.
